(19) United States
(12) Patent Application Publication
(10) Pub. No.: US 2023/0338521 A1
(43) Pub. Date: Oct. 26, 2023

(54) METHODS OF TREATING CANCER USING A COMBINATION OF A PD-1 ANTAGONIST, A CHEMORADIATION THERAPY AND A PARP INHIBITOR

(71) Applicants: Roger M. PERLMUTTER, Kenilworth, NJ (US); Maria Catherine PIETANZA, Rahway, NJ (US); Merck Sharp & Dohme LLC, Rahway, NJ (US)

(72) Inventors: Roger M. Perlmutter, Santa Barbara, CA (US); Maria Catherine Pietanza, New York, NY (US)

(73) Assignee: Merck Sharp & Dohme LLC, Rahway, NJ (US)

(21) Appl. No.: 17/920,860

(22) PCT Filed: Apr. 29, 2021
(86) PCT No.: PCT/US2021/029777

§ 371 Date: Oct. 24, 2022

Related U.S. Application Data
(60) Provisional application No. 63/019,699, filed on May 4, 2020.

(51) Int. Cl. A61K 39/395; A61K 31/502; A61P 35/00; A61K 33/243; A61K 31/519; A61K 31/7048; A61K 31/282; A61K 31/337; A61N 5/10

(52) U.S. Cl. CPC A61K 39/3955; A61K 31/502; A61P 35/00; A61K 33/243; A61K 31/519; A61K 31/7048; A61K 31/282; A61K 31/337; A61N 5/10; A61N 2005/1098

(57) ABSTRACT

Provided herein are methods of treating cancer using a combination of (a) one or more programmed death 1 protein (PD-1) antagonists, (b) a radiotherapy, (c) one or more poly (ADP-ribose) polymerase (PARP) inhibitor, and optionally, (d) one or more chemotherapies. Also provided herein is a kit for pharmaceutical administration comprising: (a) a PD-1 antagonist; (b) a radiotherapy; (c) a PARP inhibitor; and (d) optionally, a chemotherapy. Further provided herein are uses of a combination for treating cancer in a human patient, wherein the combination comprises: (a) an effective amount of one or more PD-1 antagonists, (b) an effective amount of a radiotherapy, (c) an effective amount of a PARP inhibitor, and (d) optionally, one or more chemotherapies.

### FIELD OF THE INVENTION

Provided herein are methods of treating cancer using a combination of (a) one or more programmed cell death 1 (PD-1) antagonists, (b) a radiotherapy, (c) one or more poly (ADP-ribose) polymerase (PARP) inhibitor, and optionally, (d) one or more chemotherapies.

### BACKGROUND OF THE INVENTION

PD-1 is recognized as an important player in immune regulation and the maintenance of peripheral tolerance. Immune checkpoint therapies targeting PD-1 or its ligand (e.g., PD-L1) have resulted in groundbreaking improvements in clinical response in multiple human cancer types (Brahmer et al., N Engl J Med, 366: 2455-2465 (2012); Garon et al., N Engl J Med, 372:2018-2028 (2015); Hamid et al., N Engl J Med, 369:134-144 (2013); Robert et al., Lancet, 384:1109-1117 (2014); Robert et al., N Engl J Med, 372: 2521-2532 (2015); Robert et al., N Engl J Med, 372:320-330 (2015); Topalian et al., N Engl J Med, 366:2443-2454 (2012); Topalian et al., J Clin Oncol, 32:1020-1030 (2014); Wolchok et al., N Engl J Med, 369:122-133 (2013)). Immune therapies targeting the PD-1 axis include monoclonal antibodies directed to the PD-1 receptor (e.g., KEYTRUDA® (pembrolizumab), Merck and Co., Inc., Kenilworth, NJ; OPDIVO® (nivolumab), Bristol-Myers Squibb Company, Princeton, NJ); LIBTAYO® (cemiplimab), Regeneron Pharmaceuticals, Inc., Tarrytown, NY; and those that bind to the PD-L1 ligand (e.g., TECENTRIQ® (atezolizumab), Genentech, San Francisco, CA); IMFINZI® (durvalumab), AstraZeneca Pharmaceuticals LP, Wilmington, DE; and BAVENCIO® (avelumab), Pfizer Inc., New York, NY.

The mammalian poly (ADP-ribose) polymerase (PARP) enzyme (a 113-kDa multidomain protein) has been implicated in the signalling of DNA damage through its ability to recognize and rapidly bind to DNA single or double strand breaks (D′ Amours, et al., Biochem. J., 342, 249-268 (1999)).

Several observations have led to the conclusion that PARP participates in a variety of DNA-related functions including gene amplification, cell division, differentiation, apoptosis, DNA base excision repair and also effects on telomere length and chromosome stability (dDAdda di Fagagna, et al., Nature Gen., 23(1), 76-80 (1999)).

Studies on the mechanism by which PARP modulates DNA repair and other processes has identified its importance in the formation of poly(ADP-ribose) chains within the cellular nucleus (Althaus, F. R. and Richter, C., ADP-Ribosylation of Proteins: Enzymology and Biological Significance, Springer-Verlag, Berlin (1987)). The DNA-bound, activated PARP utilizes nicotinamide adenine dinucleotide (NAD) to synthesize poly (ADP-ribose) on a variety of nuclear target proteins, including topoisomerase, 40 histones and PARP itself (Rhun, et al., Biochem. Biophys. Res. Commun., 245, 1-10 (1998).

Poly(ADP-ribosyl)ation has also been associated with malignant transformation. For example, PARP activity is higher in the isolated nuclei of SV 40-transformed fibroblasts, while both leukemic cells and colon cancer cells show higher enzyme activity than the equivalent normal leukocytes and colon mucosa (Miwa, et al., Arch. Biochem. Biophys., 181, 313-321 (1977); Burzio, et al., Proc. Soc. Exp. Bioi. Med., 149, 933-938 (1975); and Hirai, et al., Cancer Res., 43, 34413446 (1983)).

It has been proposed that the efficacy of anti-PD-1 or anti-PD-L1 antagonistic antibodies might be enhanced if administered in combination with other approved or experimental cancer therapies. However, there are no clear guidelines as to which agent combined with the PD-1 antagonist may be effective or in which patients the combination may enhance the efficacy of treatment. Thus, there is an unmet need for high efficacy therapeutic combinations that can generate a robust immune response to cancer.

### SUMMARY OF THE INVENTION

Disclosed herein are methods of treating cancer (e.g., breast cancer, ovarian cancer, non-small cell lung cancer (NSCLC), or pancreatic cancer) comprising administering to a patient in need thereof a combination of (a) an effective amount of one ore more programmed cell death 1 (PD-1) antagonists, (b) an effective amount of a radiotherapy, (c) an effective amount of a poly(ADP-ribose) polymerase (PARP) inhibitor, and optionally, (d) one or more chemotherapies.

In one embodiment, a method of treating cancer comprises administering to a patient in need thereof a combination of (a) a PD-1 antagonist, (b) a radiotherapy, (c) a PARP inhibitor, and (d) a chemotherapy, comprising:
(1) a treatment phase comprising administering an effective amount of a PD-1 antagonist in combination with an effective amount of a radiotherapy and an effective amount of a chemotherapy; and followed by
(2) a maintenance phase comprising administering an effective amount of a PD-1 antagonist in combination with an effective amount of a PARP inhibitor.

In one embodiment, a method of treating cancer comprises:
(1) a treatment phase comprising administering an effective amount of a PD-1 antagonist in combination with an effective amount of a radiotherapy and an effective amount of a chemotherapy;
wherein the PD-1 antagonist is administered once or multiple times;
wherein the radiotherapy is administered at a dose of about 20 Gy to about 80 Gy in one or more fractions; and
wherein the chemotherapy is administered once or multiple times; and followed by
(2) a maintenance phase comprising administering an effective amount of a PD-1 antagonist in combination with an effective amount of a PARP inhibitor;
wherein the PD-1 antagonist is administered once or multiple times up to 12 months; and
wherein the PARP inhibitor is administered once or multiple times up to 12 months.

Also provided herein are kits for pharmaceutical administration comprising: (a) a PD-1 antagonist; (b) a radiotherapy; (c) a PARP inhibitor; and (d) optionally, a chemotherapy.

### BRIEF DESCRIPTION OF THE DRAWINGS

FIG. 1 illustrate a schema of a clinical phase 2 study of pembrolizumab plus platinum doublet chemotherapy and concurrent radiotherapy as first-line therapy for unresectable, locally advanced stage III NSCLC (NSCLC).

FIG. 2 illustrates a schema of a clinical phase 3 study of pembrolizumab with concurrent chemoradiation therapy followed by pembrolizumab with or without olaparib in stage III non-small cell lung cancer (NSCLC).

### DETAILED DESCRIPTION OF THE INVENTION

It has been surprisingly found that using a combination of a PD-1 antagonist and a PARP inhibitor as a maintenance therapy of cancer after initial treatment of such cancer comprising administering a PD-1 antagonist, a radiotherapy, and optionally, a chemotherapy, provides improved benefits, including improved efficacy.

Disclosed herein is a method of treating cancer comprising administering to a patient in need thereof a combination of: (a) an effective amount of one or more PD-1 antagonists; (b) an effective amount of a radiotherapy; (c) an effective amount of one or more PARP inhibitors; and (d) optionally, one or more chemotherapies.

In one embodiment of the method, each PARP inhibitor of (c) is independently selected from olaparib, niraparib, rucaparib, and talazoparib, or a pharmaceutically acceptable salt thereof.

In one embodiment of the method, the PARP inhibitor is olaparib, or a pharmaceutically acceptable salt thereof. In one embodiment, the PARP inhibitor is administered once or multiple times.

Olaparib has the chemical name of 4-[3-(4-cyclopropanecarbonyl-piperazine-1-carbonyl)-4-fluoro-benzyl]-2H-phthalazin-1-one, and is represented by Formula (I),

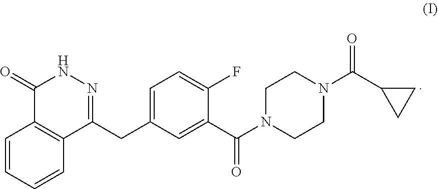 [SMILES: O=C(c1cc(Cc2n[nH]c(=O)c3ccccc23)ccc1F)N1CCN(C(=O)C2CC2)CC1]

In one embodiment of the method, the PD-1 antagonist is an anti-PD-1 antibody. In another embodiment, the PD-1 antagonist is an anti-PD-L1 antibody. In one embodiment of the method, each PD-1 antagonist of (a) is an anti-PD-1 antibody and is independently selected from pembrolizumab, nivolumab, cemiplimab, sintilimab, tislelizumab, camrelizumab and toripalimab; and each PD-L1 antagonist of (a) is an anti-PD-L1 antibody independently selected from atezolizumab, durvalumab and avelumab.

In one embodiment of the method, the PD-1 antagonist of (a) is an anti-PD-1 antibody and is selected from pembrolizumab and nivolumab and is administered once or multiple times. In one embodiment of the method, the anti-PD-1 antibody of (a) is pembrolizumab. In another embodiment, pembrolizumab is administered once or multiple times. In one embodiment of the method, the radiotherapy of (b) is a standard thoracic radiotherapy.

In one embodiment, the radiotherapy is administered at a dose of about 1 Gy to about 150 Gy in one or more fractions.

In one embodiment, the radiotherapy is administered at a dose of about 10 Gy to about 100 Gy in one or more fractions.

In one embodiment of the method, the radiotherapy of (b) is administered at a dose of about 20 Gy to about 80 Gy in one or more fractions.

In one embodiment of the method, the radiotherapy of (b) is administered at a dose of about 60 Gy in 30 daily doses of 2 Gy.

In one embodiment of the method, the one or more PD-1 antagonists of (a), the radiotherapy of (b), the one or more PARP inhibitors of (c), and the optional one or more chemotherapies of (d) are administered according to the following:
(1) a treatment phase comprising administering an effective amount of a PD-1 antagonist in combination with an effective amount of a radiotherapy and an optional chemotherapy; and followed by
(2) a maintenance phase comprising administering an effective amount of a PARP inhibitor.

In one embodiment of the method, the one or more PD-1 antagonists of (a), the radiotherapy of (b), the one or more PARP inhibitors of (c), and the optional one or more chemotherapies of (d) are administered according to the following:
(1) a treatment phase comprising administering an effective amount of a PD-1 antagonist in combination with an effective amount of a radiotherapy and an optional chemotherapy; and followed by
(2) a maintenance phase comprising administering an effective amount of a PD-1 antagonist in combination with an effective amount of a PARP inhibitor.

In one embodiment, the method comprises:
(1) a treatment phase comprising administering an effective amount of a PD-1 antagonist in combination with the radiotherapy and an effective amount of a chemotherapy;
wherein the radiotherapy and the chemotherapy are administered concurrently; and followed by:
(2) a maintenance phase comprising administering an effective amount of a PD-1 antagonist in combination with an effective amount of the PARP inhibitor;
wherein the PD-1 antagonist is administered once or multiple times up to 12 months; and
wherein the PARP inhibitor is administered once or multiple times up to 12 months.

In one embodiment of the method, the PD-1 antagonist is an anti-PD-1 antibody. In another embodiment, the PD-1 antagonist is an anti-PD-L1 antibody. In one embodiment of the method, each PD-1 antagonist of the treatment phase (1) is an anti-PD-1 antibody and is selected from pembrolizumab and nivolumab; and each PD-1 antagonist of the maintenance phase (2), when present, is an anti-PD-1 antibody and is selected from pembrolizumab and nivolumab.

In one embodiment of the method, each anti-PD-1 antibody of the treatment phase (1) is pembrolizumab; each anti-PD-1 antibody of (2), when present, is pembrolizumab; the radiotherapy of (b) is a standard thoracic radiotherapy administered at a dose of about 20 to about 80 Gy in multiple fractions; and each PARP inhibitor of (c) is olaparib, or a pharmaceutically acceptable salt thereof.

In one embodiment of the method, the optional chemotherapy is administered. In one embodiment of the method, the chemotherapy is selected from adriamycin, bleomycin, cisplatin, carboplatin, dactinomycin, daunorubicin, docetaxel, etoposide, irinotecan, mitomycin C, paclitaxel, pemetrexed, plicamycin, podophyllotoxin, topotecan, vincristine, and a combination of any two or more of the forgoing chemotherapies.

In one embodiment of the method, the chemotherapy is selected from cisplatin, carboplatin, etoposide, paclitaxel, pemetrexed and a combination of any two of the foregoing chemotherapies.

In one embodiment of the method, the chemotherapy is a platinum doublet selected from:
(1) a combination of cisplatin and pemetrexed;
(2) a combination of cisplatin and etoposide; and
(3) a combination of carboplatin and paclitaxel.

In one embodiment of the method, the chemotherapy is a platinum doublet selected from:
(1) cisplatin 75 mg/m² IV and pemetrexed 500 mg/m² IV in three cycles (Day 1 in each cycle of Cycles 1-3);
(2) cisplatin 50 mg/m² IV (Days 1 and 8 in Cycles 1 and 2; Days 8 and 15 in Cycle 3) and etoposide 50 mg/m² IV in three cycles (Days 1 to 5 in Cycles 1 and 2; days 8 to 12 in Cycle 3); and
(3) carboplatin AUC 6 mg/mL/min IV with paclitaxel 200 mg/m² IV on Day 1 in Cycle 1; carboplatin AUC 2 mg/mL/min IV with paclitaxel 45 mg/m² IV on Days 1, 8, and 15 in Cycles 2 and 3.

In one embodiment, the method comprises administering a PD-1 antagonist of (a), a radiotherapy of (b), a PARP inhibitor of (c) and a chemotherapy of (d) according to the following:
(1) a treatment phase comprising administering an effective amount of a PD-1 antagonist in combination with an effective amount of a radiotherapy and an effective amount of a chemotherapy; and followed by
(2) a maintenance phase comprising administering an effective amount of a PD-1 antagonist in combination with an effective amount of a PARP inhibitor.

In one embodiment, the method comprises:
(1) a treatment phase comprising administering an effective amount of a PD-1 antagonist in combination with an effective amount of a radiotherapy and an effective amount of a chemotherapy;
wherein the PD-1 antagonist is administered once or multiple times;
wherein the radiotherapy is at a dose of about 20 Gy to about 80 Gy in one or multiple fractions; and
wherein the chemotherapy is administered once or multiple times; and followed by
(2) a maintenance phase comprising administering an effective amount of a PD-1 antagonist in combination with an effective amount of a PARP inhibitor;
wherein the PD-1 antagonist is administered once or multiple times up to 12 months; and
wherein the PARP inhibitor is administered once or multiple times up to 12 months.

In one embodiment, the method comprises:
(1) a treatment phase comprising administering an effective amount of a PD-1 antagonist in combination with an effective amount of a radiotherapy and an effective amount of a chemotherapy;
wherein the PD-1 antagonist an anti-PD-1 antibody and is selected from pembrolizumab, nivolumab, cemiplimab, sintilimab, tislelizumab, camrelizumab and toripalimab;
wherein the radiotherapy is a standard thoracic radiotherapy; and
wherein the chemotherapy is selected from adriamycin, bleomycin, cisplatin, carboplatin, dactinomycin, daunorubicin, docetaxel, etoposide, irinotecan, mitomycin C, paclitaxel, pemetrexed, plicamycin, podophyllotoxin, topotecan, vincristine and a combination of two of the foregoing chemotherapies; and followed by
(2) a maintenance phase comprising administering an effective amount of a PD-1 antagonist in combination with an effective amount of a PARP inhibitor;
wherein the PD-1 antagonist is an anti-PD-1 antibody and is selected from pembrolizumab, nivolumab, cemiplimab, sintilimab, tislelizumab, camrelizumab and toripalimab; and
wherein the PARP inhibitor is selected from olaparib, niraparib, rucaparib, and talazoparib, or a pharmaceutically acceptable salt thereof.

In one embodiment of the method, the PD-1 antagonist of the treatment phase (1) is administered at a dose of 50 mg to 600 mg or 1-4 mg/kg once every three to six weeks.

In one embodiment of the method, the PD-1 antagonist of the treatment phase (1) is pembrolizumab administered at a dose of 200 mg or 2 mg/kg IV once every three weeks.

In one embodiment of the method, the PD-1 antagonist of the treatment phase (1) is pembrolizumab administered at a dose of 400 mg or 4 mg/kg IV once every six weeks.

In one embodiment of the method, the PD-1 antagonist of the treatment phase (1) is nivolumab and is administered at a dose of 240 mg IV once every two weeks.

In one embodiment of the method, the PD-1 antagonist of the treatment phase (1) is nivolumab and is administered at a dose of 480 mg IV once every four weeks.

In one embodiment of the method, the PD-1 antagonist of the treatment phase (1) is cemiplimab and is administered at a dose of 350 mg IV every three weeks.

In one embodiment of the method, the radiotherapy is administered at a dose of about 20 Gy to about 80 Gy.

In one embodiment of the method, the radiotherapy is administered at a dose of about 60 Gy in fractions of 30 daily doses of 2 Gy.

In one embodiment of the method, the chemotherapy is selected from cisplatin, carboplatin, etoposide, paclitaxel, pemetrexed and a combination of any two of the foregoing chemotherapies.

In one embodiment of the method, the chemotherapy is selected from cisplatin, carboplatin, etoposide, paclitaxel, pemetrexed and a combination of any two of the foregoing chemotherapies; and administered at a dose of 10-2000 mg/m² of each chemotherapy up to 3 cycles.

In one embodiment of the method, the chemotherapy is selected from:
(1) cisplatin 75 mg/m² IV and pemetrexed 500 mg/m² IV in three cycles (Day 1 in each cycle of Cycles 1-3);
(2) cisplatin 50 mg/m² IV (Days 1 and 8 in Cycles 1 and 2; Days 8 and 15 in Cycle 3) and etoposide 50 mg/m² IV in three cycles (Days 1 to 5 in Cycles 1 and 2; days 8 to 12 in Cycle 3); and
(3) carboplatin AUC 6 mg/mL/min IV with paclitaxel 200 mg/m² IV on Day 1 in Cycle 1; carboplatin AUC 2 mg/mL/min IV with paclitaxel 45 mg/m² IV on Days 1, 8, and 15 in Cycles 2 and 3.

In one embodiment of the method, the PD-1 antagonist of the maintenance phase (2) is administered at a dose of 100 mg to 600 mg once every three to six weeks.

In one embodiment of the method, the PD-1 antagonist of the maintenance phase (2) is pembrolizumab and is administered at a dose of 200 mg once every three weeks for up to 12 months.

In one embodiment of the method, the PD-1 antagonist of the maintenance phase (2) is pembrolizumab and is administered at a dose of 200 mg or 2 mg/kg IV once every three weeks for up to 12 months.

In one embodiment of the method, the PD-1 antagonist of the maintenance phase (2) is pembrolizumab and is administered at a dose of 200 mg IV once every three weeks for up to 12 months.

In one embodiment of the method, the PD-1 antagonist of the maintenance phase (2) is pembrolizumab and is administered at a dose of 400 mg once every three weeks for up to 12 months.

In one embodiment of the method, the PD-1 antagonist of the maintenance phase (2) is pembrolizumab administered at a dose of 400 mg or 4 mg/kg IV once every six weeks for up to 12 months.

In one embodiment of the method, the pembrolizumab is administered at a dose of 400 mg once every six weeks for up to 12 months.

In one embodiment of the method, the PARP inhibitor of the maintenance phase (2) is administered at a dose of 100 mg to 600 mg twice daily.

In one embodiment of the method, the PARP inhibitor of the maintenance phase (2) is olaparib, or a pharmaceutically acceptable salt thereof, and is administered at a dose of 300 mg twice daily.

In one embodiment of the method, the olaparib, or a pharmaceutically acceptable salt thereof, is administered at a dose of 300 mg twice daily for up to 12 months.

In one embodiment, a method comprises:
(1) a treatment phase comprising administering a PD-1 antagonist in combination with a radiotherapy and a chemotherapy;
wherein the PD-1 antagonist is administered once every three to six weeks;
wherein the radiotherapy is administered at a dose of about 20 Gy to about 80 Gy in one or more fractions;
wherein the chemotherapy is selected from cisplatin, carboplatin, etoposide, paclitaxel, pemetrexed and a combination of any two of the foregoing chemotherapies; and is administered at a dose of 10-200 mg/m² of each chemotherapy up to 3 cycles; and followed by
(2) a maintenance phase comprising administering a PD-1 antagonist in combination with a PARP inhibitor;
wherein the PD-1 antagonist is administered once every three to six weeks in one or more cycles; and
wherein the PARP inhibitor is administered at a dose of 100 mg to 600 mg twice daily in one or more cycles.

In one embodiment of the method, the PD-1 antagonist of the treatment phase (1) and/or the maintenance phase (2) is administered at a dose of 100 mg to 600 mg once every three to six weeks.

In one embodiment, a method comprises:
(1) a treatment phase comprising administering a PD-1 antagonist in combination with a radiotherapy and a chemotherapy;
wherein the PD-1 antagonist is pembrolizumab administered at a dose of 200 mg once every three weeks;
wherein the radiotherapy is a standard thoracic radiotherapy administered at a dose of about 60 Gy in fractions of 30 daily doses of 2 Gy;
wherein the chemotherapy is selected from cisplatin, carboplatin, etoposide, paclitaxel, pemetrexed and a combination of any two of the foregoing chemotherapies; and is administered at a dose of 10-200 mg/m² of each chemotherapy up to 3 cycles; and followed by
(2) a maintenance phase comprising administering a PD-1 antagonist in combination with a PARP inhibitor;
wherein the PD-1 antagonist is pembrolizumab administered at a dose of 200 mg once every three weeks for up to 12 months; and
wherein the PARP inhibitor is olaparib, or a pharmaceutically acceptable salt thereof, administered at a dose of 300 mg twice daily for up to 12 months.

In one embodiment of the method, the PD-1 antagonist of the treatment phase (1) and/or the maintenance phase (2) is an anti-PD-1 antibody.

In one embodiment, the above method comprises:
(1) a treatment phase comprising administering a PD-1 antagonist in combination with a radiotherapy and a chemotherapy;
wherein the PD-1 antagonist is pembrolizumab administered at a dose of 400 mg once every six weeks;
wherein the radiotherapy is a standard thoracic radiotherapy administered at a dose of about 60 Gy in fractions of 30 daily doses of 2 Gy;
wherein the chemotherapy is selected from cisplatin, carboplatin, etoposide, paclitaxel, pemetrexed and a combination of any two of the foregoing chemotherapies; and administered at a dose of 10-200 mg/m² of each chemotherapy up to 3 cycles; and followed by
(2) a maintenance phase comprising administering a PD-1 antagonist which is an anti-PD-1 antibody in combination with a PARP inhibitor;
wherein the PD-1 antagonist is pembrolizumab administered at a dose of 400 mg once every six weeks for up to 12 months; and
wherein the PARP inhibitor is olaparib, or a pharmaceutically acceptable salt thereof, administered at a dose of 300 mg twice daily for up to 12 months.

In one embodiment of the method, the PD-1 antagonist of the treatment phase (1) and/or the maintenance phase (2) is an anti-PD-1 antibody.

In one embodiment of the method, the PD-1 antagonist, the radiotherapy and the chemotherapy of the treatment phase (1) are concurrent therapies administered on the same day or on different days, and are administered sequentially or concurrently.

In one embodiment of the method, the PD-1 antagonist and the PARP inhibitor of the maintenance phase (2) are administered on the same day or on different days, and are administered sequentially or concurrently.

In one embodiment of the method, the PD-1 antagonist and the PARP inhibitor of the maintenance phase (2) are administered on the same day or on different days, and are administered sequentially.

In one embodiment, provided herein is a kit for pharmaceutical administration comprising:
(a) a PD-1 antagonist;
(b) instructions on administering a radiotherapy;
(c) a PARP inhibitor; and
(d) optionally, a chemotherapy.

In one embodiment, a kit further comprises instructions for administering to a human patient (a) the PD-1 antagonist, (b) the radiotherapy, (c) the PARP inhibitor, and optionally, (d) the chemotherapy.

In one embodiment, provided herein is a kit for pharmaceutical administration comprising:
(a) a PD-1 antagonist selected from pembrolizumab, nivolumab, cemiplimab, sintilimab, tislelizumab, camrelizumab and toripalimab;
(b) instructions on administering a radiotherapy as part of a treatment phase;
(c) a PARP inhibitor selected from olaparib, niraparib, rucaparib, and talazoparib, or a pharmaceutically acceptable salt thereof; and
(d) a chemotherapy selected from cisplatin, carboplatin, etoposide, paclitaxel, pemetrexed and a combination of any two of the foregoing chemotherapies.

In one embodiment, provided herein is a kit for pharmaceutical administration comprising:
(a) a PD-1 antagonist which is pembrolizumab;
(b) instructions on administering a radiotherapy as part of a treatment at a dose of about 20 Gy to about 80 Gy;
(c) a PARP inhibitor which is olaparib, or a pharmaceutically acceptable salt thereof; and
(d) a chemotherapy selected from:
   (1) a combination of cisplatin and pemetrexed;
   (2) a combination of cisplatin and etoposide; and
   (3) a combination of carboplatin and paclitaxel.

In one embodiment of the kit, the kit further comprises f instructions for administering to a human patient (a) the PD-1 antagonist, (b) the radiotherapy, (c) the PARP inhibitor, and optionally, (d) the chemotherapy.

In one embodiment of the method, the cancer is selected from the group consisting of bladder cancer, breast cancer, colorectal cancer, hepatocellular carcinoma, melanoma, non-small cell lung cancer (NSCLC), ovarian cancer, pancreatic cancer, prostate cancer, and renal cell carcinoma.

In one embodiment of the method, the cancer is NSCLC.

In one embodiment of the method, the cancer is unresectable, locally advanced, Stage III NSCLC.

In one embodiment, the cancer is metastatic.

In another embodiment, the cancer is relapsed.

In another embodiment, the cancer is refractory.

In yet another embodiment, the cancer is relapsed and refractory.

In one embodiment, the cancer is bladder cancer.

In another embodiment, the cancer is breast cancer.

In another embodiment, the cancer is colorectal cancer.

In another embodiment, the cancer is hepatocellular carcinoma.

In another embodiment, the cancer is melanoma.

In another embodiment, the cancer is non-small cell lung cancer (NSCLC).

In another embodiment, the cancer is ovarian cancer.

In another embodiment, the cancer is pancreatic cancer.

In another embodiment, the cancer is prostate cancer.

In another embodiment, the cancer is renal cell carcinoma.

In one embodiment of the methods or kits provided herein, the PD-1 antagonist is an anti-human PD-1 monoclonal antibody or antigen binding fragment thereof. In another embodiment, the PD-1 antagonist is an anti-human PD-L1 monoclonal antibody or antigen binding fragment thereof.

In one embodiment of the methods or kits provided herein, the anti-human PD-1 monoclonal antibody is a humanized antibody.

In one embodiment of the methods or kits provided herein, the anti-human PD-L1 monoclonal antibody is a humanized antibody.

In one embodiment of the methods or kits provided herein, the anti-human PD-1 monoclonal antibody is a human antibody.

In one embodiment of the methods or kits provided herein, the anti-human PD-L1 monoclonal antibody is a human antibody.

In one embodiment of the methods or kits provided herein, the anti-human PD-1 monoclonal antibody is pembrolizumab.

In one embodiment of the methods or kits provided herein, the anti-human PD-1 monoclonal antibody is nivolumab.

In one embodiment of the methods or kits provided herein, the anti-human PD-1 monoclonal antibody is cemiplimab.

In one embodiment of the methods or kits provided herein, the anti-human PD-L1 monoclonal antibody is atezolizumab.

In one embodiment of the methods or kits provided herein, the anti-human PD-L1 monoclonal antibody is durvalumab.

In one embodiment of the methods or kits provided herein, the anti-human PD-L1 monoclonal antibody is avelumab.

In one embodiment of the methods or kits provided herein, the human patient is administered 200 mg, or 2 mg/kg pembrolizumab, and pembrolizumab is administered once every three weeks. In one embodiment, the human patient is administered 200 mg pembrolizumab once every three weeks. In one embodiment, the human patient is administered 2 mg/kg pembrolizumab once every three weeks.

In one embodiment of the methods or kits provided herein, the human patient is administered 240 mg or 3 mg/kg nivolumab once every two weeks, or 480 mg nivolumab once every four weeks. In one embodiment, the human patient is administered 240 mg nivolumab once every two weeks. In one embodiment, the human patient is administered 3 mg/kg nivolumab once every two weeks. In one embodiment, the human patient is administered 480 mg nivolumab once every four weeks.

In one embodiment of the methods or kits provided herein, the human patient is administered 350 mg cemiplimab, and cemiplimab is administered once every three weeks.

In one embodiment of the methods or kits provided herein, the human patient is administered 800 mg of avelumab, and aveulmab is administered once every two weeks.

In one embodiment of the methods or kits provided herein, the human patient is administered 840 mg of atezolizumab, and the atezolizumab is administered once every two weeks. In one embodiment, the human patient is administered 1200 mg of atezolizumab, and the atezolizumab is administered once every three weeks. In one embodiment, the human patient is administered 1680 mg of atezolizumab, and the atezolizumab is administered once every four weeks.

In one embodiment of the methods or kits provided herein, the human patient is administered 10 mg/kg of durvalumab, and the durvalumab is administered once every two weeks. In one embodiment, the human patient is administered 1500 mg of durvalumab, and the durvalumab is administered once every three weeks. In another embodiment, the human patient is administered 1500 mg of durvalumab, and the durvalumab is administered once every four weeks.

In yet still other embodiments of various methods described herein, the human patient is administered 100, 150, 200, 250, 300, 350, 400, 450, 500 or 550 mg olaparib, or a pharmaceutically acceptable salt thereof, twice daily.

Thus, in one embodiment, a human patient is administered:
during a treatment phase:
(a) 200 mg, 240 mg, or 2 mg/kg pembrolizumab once every three weeks;
(b) a radiotherapy at a dose of about 60 Gy in 30 daily doses of 2 Gy;
(c) a chemotherapy once every three weeks in 3 cycles; and followed by a maintenance phase comprising administering:

(d) 200 mg, 240 mg, or 2 mg/kg pembrolizumab once every three weeks for up to 12 months;
(e) 400 mg olaparib twice daily for up to 12 months.

### Definitions

Certain technical and scientific terms are specifically defined below. Unless specifically defined elsewhere in this document, all other technical and scientific terms used herein have the meaning commonly understood by one of ordinary skill in the art to which this disclosure relates.

“About” when used to modify a numerically defined parameter (e.g., the dose of an anti-PD-1 antibody or antigen binding fragment thereof, olaparib, radiotherapy or chemotherapy, or the length of treatment time with a combination therapy described herein) means that the parameter is within 10%, or less of the stated numerical value or range for that parameter; where appropriate, the stated parameter may be rounded to the nearest whole number. For example, a dose of about 5 mg/kg may vary between 4.5 mg/kg and 5.5 mg/kg.

As used herein, including the appended claims, the singular forms of words such as “a,” “an,” and “the,” include their corresponding plural references unless the context clearly dictates otherwise.

The terms “administration” or “administer” refers to the act of injecting or otherwise physically delivering a substance as it exists outside the body (e.g., an anti-PD-1 antibody, olaparib, chemotherapy, or radiation, as described herein) into a patient, such as by oral, mucosal, intradermal, intravenous, subcutaneous, intramuscular delivery, and/or any other methods of physical delivery described herein or known in the art.

The term “effective amount” refers to the amount of a compound that will elicit a biological or medical response of a tissue, system, animal or human that is being sought by a researcher, veterinarian, medical doctor or other clinician when administered to a patient such as a human patient. An effective amount does not necessarily include considerations of toxicity and safety related to the administration of a compound. It is recognized that one skilled in the art may affect physiological disorders associated with a PD-1 enzyme activity or a PARP enzyme activity by treating a patient presently afflicted with the disorders, or by prophylactically treating a patient likely to be afflicted with the disorders, with an effective amount of a compound disclosed herein, or a pharmaceutically acceptable salt, solvate or hydrate thereof. As applied to a radiotherapy or a chemotherapy, the term “effective amount” refers to a dose of the radiotherapy or chemotherapy that will elicit a biological or medical response of a tissue, system, animal or human that is being sought by a researcher, veterinarian, medical doctor or other clinician when administering the radiotherapy or chemotherapy to a patient such as a human patient.

An effective amount further refers to the amount of the compound or therapy that, when administered alone or in combination to a cell, tissue, or subject, is effective to cause a measurable improvement in one or more symptoms of a cancer or the progression of cancer. An effective amount further refers to that amount of the compound or therapy, alone or in combination, sufficient to result in at least partial amelioration of symptoms, e.g., tumor shrinkage or elimination, lack of tumor growth, increased survival time. An effective amount may result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. Toxicity and therapeutic efficacy of the compounds, therapies, combinations and compositions of the invention, administered alone or in combination, can be determined by any number of systems or means. For example, the toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD50/ED50). The data obtained from these cell culture assays and animal studies can be used in formulating a range of dosage for use in human. The dosage of such compounds or therapies, alone or in combination, lies preferably within a range of circulating concentrations that include the ED50 with little or no toxicity. The dosage may vary within this range depending upon the dosage form employed and the route of administration.

“PD-1 antagonist” means any chemical compound or biological molecule that blocks binding of PD-L1 expressed on cell (e.g., cancer cell) to PD-1 expressed on a different cell (e.g., an immune cell such as a T cell, B cell or Natural Killer T (NKT cell) and preferably also blocks binding of PD-L2 expressed on a cell (e.g., a cancer cell) to the cell expressing PD-1 (e.g., the immune-cell expressed PD-1). Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and disclosed uses in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively. The term PD-1 antagonist includes anti-PD-1 antibodies and anti-PD-L1 antibodies.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} β-sheet framework, or one of three hypervariable regions (L1, L2, or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J Mol. Biol. 222: 581-597, for example. See also Presta (2005) J Allergy Clin. Immunol. 116:731.

As used herein, unless otherwise indicated, “antibody fragment” or “antigen binding fragment” refers to a fragment of an antibody that retains the ability to bind specifically to the antigen, e.g., fragments that retain one or more CDR regions. An antibody that “specifically binds to” PD-1 is an antibody that exhibits preferential binding to PD-1 as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., PD-1). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, the terms “at least one” item or “one or more” item each include a single item selected from the list as well as mixtures of two or more items selected from the list.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

The term “subject” (alternatively “patient”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorphs (e.g., rabbits), rodents (e.g., rats or mice), Procyon lotor (e.g., raccoons). In particular embodiments, the subject is human.

The term “subject in need thereof” as used herein refers to a subject diagnosed with or suspected of having cancer as defined herein.

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient, and can be readily selected by a person skilled in the art.

The term “variant” when used in relation to an antibody (e.g., an anti-PD-1 antibody) or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified anti-PD-1 antibody. Variants may be naturally occurring or may be artificially constructed. Polypeptide variants may be prepared from the corresponding nucleic acid molecules encoding the variants. In specific embodiments, an antibody variant (e.g., an anti-PD-1 antibody variant) at least retains the antibody functional activity. In specific embodiments, an anti-PD-1 antibody variant binds to PD-1 and/or is antagonistic to PD-1 activity.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“RECIST 1.1 Response Criteria” as used herein means the definitions set forth in Eisenhauer, E. A. et al., Eur. J. Cancer 45:228-247 (2009) for target lesions or nontarget lesions, as appropriate based on the context in which response is being measured.

“Sustained response” means a sustained therapeutic effect after cessation of treatment as described herein. In some embodiments of the methods, the sustained response has a duration that is at least the same as the treatment duration, or at least 1.5, 2.0, 2.5 or 3 times longer than the treatment duration.

“Treat” or “treating” cancer as used herein means to administer a therapeutic combination of a PD-1 antagonist (e.g., an anti-human PD-1 monoclonal antibody or antigen binding fragment thereof), a radiotherapy, a poly(ADP-ribose) polymerase (PARP) inhibitor (e.g., olaparib or a pharmaceutically acceptable salt thereof), and optionally a chemotherapy, to a subject having cancer or diagnosed with cancer to achieve at least one positive therapeutic effect, such as, for example, reduced number of cancer cells, reduced tumor size, reduced rate of cancer cell infiltration into peripheral organs, or reduced rate of tumor metastasis or tumor growth. Such “treatment” may result in a slowing, interrupting, arresting, controlling, or stopping of the progression of cancer as described herein but does not necessarily indicate a total elimination of the cancer or the symptoms of the cancer. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments of the methods, the treatment achieved by a combination therapy of the disclosure is any of partial response (PR), complete response (CR), overall response (OR), progression-free survival (PFS), disease-free survival (DFS), and overall survival (OS). PFS, also referred to as “Time to Tumor Progression” indicates the length of time during and after treatment that the cancer does not grow, and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naive or untreated individuals or patients. In some embodiments, response to a combination therapy of the disclosure is any of PR, CR, PFS, DFS, or OR that is assessed using RECIST 1.1 response criteria. The treatment regimen for a combination therapy of the disclosure that is effective to treat a cancer patient may vary according to factors such as the disease state, age, and weight of the patient, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chit-test, the U-test according to Mann and Whitney, the Kruskal-Wallis test (H-test), Jonckheere-Terpstra-test and the Wilcoxon-test.

As used herein, the terms “combination,” “combination therapy,” and “therapeutic combination” refer to treatments in which at least one PD-1 antagonist (e.g., an anti-human PD-1 monoclonal antibody or antigen-binding fragment thereof), at least one radiotherapy, a poly(ADP-ribose) polymerase (PARP) inhibitor (e.g., olaparib or a pharmaceutically acceptable salt thereof), and optionally, one chemotherapy, each are administered to a patient in a coordinated manner, over an overlapping period of time.

The period of treatment with the at least one PD-1 antagonist (e.g., an anti-human PD-1 monoclonal antibody or antigen-binding fragment thereof) (the “PD-1 antagonist treatment”) is the period of time that a patient undergoes treatment with the anti-human PD-1 monoclonal antibody (or antigen-binding fragment thereof) or with the anti-human PD-L1 monoclonal antibody (or antigen binding fragment thereof); that is, the period of time from the initial dosing with the anti-human PD-1 monoclonal antibody (or antigen-binding fragment thereof) or the anti-PD-L1 monoclonal antibody (or antigen binding fragment thereof) through the final day of a treatment cycle.

The period of treatment with the radiotherapy is the period of time that a patient undergoes treatment with the radiotherapy; that is, the period of time from the initial treatment with the radiotherapy through the final day of a treatment cycle.

Similarly, the period of treatment with the chemotherapy is the period of time that a patient undergoes treatment with the chemotherapy; that is, the period of time from the initial treatment with the chemotherapy through the final day of a treatment cycle.

The period of treatment with a poly(ADP-ribose) polymerase (PARP) inhibitor (e.g., olaparib or a pharmaceutically acceptable salt thereof) (the “PARP inhibitor treatment”) is the period of time that a patient undergoes treatment with the PARP inhibitor; that is, the period of time from the initial dosing with the PARP inhibitor through the final day of a treatment cycle.

In the methods and therapeutic combinations described herein, the methods generally comprise a treatment phase followed by a maintenance phase.

During the treatment phase, the PD-1 antagonist treatment (e.g., treatment with an anti-human PD-1 monoclonal antibody or antigen binding fragment thereof) overlaps by at least one day with the radiotherapy treatment; overlaps by at least one day with the chemotherapy treatment when a chemotherapy is administered; and overlaps by at least one day with the PARP inhibitor treatment. In certain embodiments of the methods, the PD-1 antagonist, the radiotherapy, the optional chemotherapy, and the PARP inhibitor treatment are the same period of time. In some embodiments, the PD-1 antagonist treatment begins prior to the radiotherapy, and/or the optional chemotherapy. In other embodiments, the PD-1 antagonist treatment begins after the radiotherapy and/or the optional chemotherapy. In yet other embodiments, the radiotherapy and/or the optional chemotherapy begins prior to the PD-1 antagonist treatment. In certain embodiments, the PD-1 antagonist treatment is terminated prior to termination of the radiotherapy and/or the optional chemotherapy. In other embodiments, the PD-1 antagonist treatment is terminated after termination of the radiotherapy and/or the optional chemotherapy.

During the maintenance phase, the PD-1 antagonist treatment overlaps by at least one day with the PARP inhibitor treatment. In certain embodiments, the PD-1 antagonist treatment and the PARP inhibitor treatment are the same period of time. In some embodiments, the PD-1 antagonist treatment begins prior to the PARP inhibitor treatment. In other embodiments, the PD-1 antagonist treatment begins after the PARP inhibitor treatment.

The terms “treatment regimen,” “dosing protocol,” and “dosing regimen” are used interchangeably to refer to the dose and timing of administration of each therapeutic agent in a combination therapy of the disclosure.

“Tumor” as it applies to a subject diagnosed with, or suspected of having, a cancer refers to a malignant or potentially malignant neoplasm or tissue mass of any size, and includes primary tumors and secondary neoplasms. Non-limiting examples of tumors include solid tumor (e.g., sarcoma (such as chondrosarcoma), carcinoma (such as colon carcinoma), blastoma (such as hepatoblastoma), etc.) and blood tumor (e.g., leukemia (such as acute myeloid leukemia (AML)), lymphoma (such as DLBCL), multiple myeloma (MM), etc.).

The term “tumor volume” or “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MM scans.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, ±10% and their numerical equivalents. All ranges also are intended to include all included sub-ranges, although not necessarily explicitly set forth. For example, a range of 3 to 7 days is intended to include 3, 4, 5, 6, and 7 days. In addition, the term “or,” as used herein, denotes alternatives that may, where appropriate, be combined; that is, the term “or” includes each listed alternative separately as well as their combination.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

### PD-1 Antagonist

Provided herein are PD-1 antagonists that can be used in the various methods and kits disclosed herein, including any chemical compound or biological molecule that blocks binding of PD-L1 to PD-1 and preferably also blocks binding of PD-L2 to PD-1.

Any monoclonal antibodies that bind to a PD-1 polypeptide, a PD-1 polypeptide fragment, a PD-1 peptide, or a PD-1 epitope and block the interaction between PD-1 and its ligand PD-L1 or PD-L2 can be used. In some embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1. In other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L2. In yet other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1 and the interaction between PD-1 and PD-L2.

Any monoclonal antibodies that bind to a PD-L1 polypeptide, a PD-L1 polypeptide fragment, a PD-L1 peptide, or a PD-L1 epitope and block the interaction between PD-L1 and PD-1 can also be used.

In certain embodiments, the anti-human PD-1 monoclonal antibody is selected from the group consisting of pembrolizumab, nivolumab, cemiplimab, sintilimab, tislelizumab, camrelizumab, toripalimab, pidilizumab (U.S. Pat. No. 7,332,582), AMP-514 (MedImmune LLC, Gaithersburg, MD), PDR001 (U.S. Pat. No. 9,683,048), BGB-A317 (U.S. Pat. No. 8,735,553), and MGA012 (MacroGenics, Rockville, MD). In one embodiment, the anti-human PD-1 monoclonal antibody is pembrolizumab. In another embodiment, the anti-human PD-1 monoclonal antibody is nivolumab. In another embodiment, the anti-human PD-1 monoclonal antibody is cemiplimab. In yet another embodiment, the anti-human PD-1 monoclonal antibody is pidilizumab. In one embodiment, the anti-human PD-1 monoclonal antibody is AMP-514. In another embodiment, the anti-human PD-1 monoclonal antibody is PDR001. In yet another embodiment, the anti-human PD-1 monoclonal antibody is BGB-A317. In still another embodiment, the anti-human PD-1 monoclonal antibody is MGA012.

In some embodiments, the anti-human PD-1 monoclonal antibody can be any antibody, antigen binding fragment thereof, or variant thereof disclosed in U.S. Pat. Nos. 7,488,802, 7,521,051, 8,008,449, 8,354,509, 8,168,757, WO2004/004771, WO2004/072286, WO2004/056875, US2011/0271358, and WO 2008/156712, the disclosures of which are incorporated by reference herein in their entireties.

Examples of monoclonal antibodies that bind to human PD-L1 that can be used in various methods, kits, and uses described herein are disclosed in U.S. Pat. No. 8,383,796, the disclosures of which are incorporated by reference herein in their entireties. Specific anti-human PD-L1 monoclonal antibodies useful as the PD-1 antagonist in the various methods, kits, and uses described include atezolizumab, durvalumab, avelumab, and BMS-936559.

Other PD-1 antagonists useful in various methods, kits, and uses described herein include an immunoadhesion molecule that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342, the disclosures of which are incorporated by reference herein in their entireties. Specific fusion proteins useful as the PD-1 antagonist in various methods, kits, and uses described herein include AMP-224 (also known as B7-DCIg), which is a PD-L2-Fc fusion protein and binds to human PD-1.

In various embodiments, the anti-human PD-1 or anti-human PD-L1 monoclonal antibody or antigen binding fragment thereof comprises a variant of the amino acid sequences of the anti-human PD-1 or anti-human PD-L1 antibodies described herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human PD-1 or anti-human PD-L1 monoclonal antibody or antigen binding fragment thereof has a V_{L} domain with at least 95%, 90%, 85%, 80%, 75% or 50% sequence homology to one of the V_{L} domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a V_{H} domain with at least 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has a V_{L} domain with at least 95%, 90%, 85%, 80%, 75% or 50% sequence homology to one of the V_{L} domains of the anti-human PD-1 or anti-human PD-L1 antibodies described herein and a V_{H} domain with at least 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1.

In one embodiment, the anti-human PD-1 or anti-human PD-L1 monoclonal antibody or antigen binding fragment thereof has a V_{L} domain having up to 1, 2, 3, 4, 5 or more amino acid substitutions, deletions and/or additions in one of the V_{L} domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a V_{H} domain having up to 1, 2, 3, 4, 5 or more amino acid substitutions, deletions, and/or additions in one of the V_{H} domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has a V_{L} domain having up to 1, 2, 3, 4, 5 or more amino acid substitutions, deletions, and/or additions in one of the V_{L} domains of the anti-human PD-1 or anti-human PD-L1 antibodies described herein and a V_{H} domain having up to 1, 2, 3, 4, 5 or more amino acid substitutions, deletions, and/or additions in one of the V_{H} domains of the anti-human PD-1 or anti-human PD-L1 antibodies described herein, and exhibits specific binding to PD-1 or PD-L1.

In various embodiments, the anti-human PD-1 or anti-human PD-L1 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-human PD-1 or anti-human PD-L1 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-human PD-1 or anti-human PD-L1 monoclonal antibody is a human antibody. In other embodiments, the anti-human PD-1 or anti-human PD-L1 monoclonal antibody is a humanized antibody.

In some embodiments, the light chain of the anti-human PD-1 or anti-human PD-L1 monoclonal antibody has a human kappa backbone. In other embodiments, the light chain of the anti-human PD-1 or anti-human PD-L1 monoclonal antibody has a human lambda backbone.

In some embodiments, the heavy chain of the anti-human PD-1 or anti-human PD-L1 monoclonal antibody has a human IgG1 backbone. In other embodiments, the heavy chain of the anti-human PD-1 or anti-human PD-L1 monoclonal antibody has a human IgG2 backbone. In yet other embodiments, the heavy chain of the anti-human PD-1 or anti-human PD-L1 monoclonal antibody has a human IgG3 backbone. In still other embodiments, the heavy chain of the anti-human PD-1 or anti-human PD-L1 monoclonal antibody has a human IgG4 backbone.

In some embodiments, the heavy chain of the anti-human PD-1 or anti-human PD-L1 monoclonal antibody has a human IgG1 variant backbone. In other embodiments, the heavy chain of the anti-human PD-1 or anti-human PD-L1 monoclonal antibody has a human IgG2 variant backbone. In yet other embodiments, the heavy chain of the anti-human PD-1 or anti-human PD-L1 monoclonal antibody has a human IgG3 variant backbone. In still other embodiments, the heavy chain of the anti-human PD-1 or anti-human PD-L1 monoclonal antibody has a human IgG4 variant (e.g., IgG4 with S228P mutation) backbone.

### Radiotherapy

The term “radiotherapy” or “radiation therapy” as provided herein for the various methods and kits disclosed herein refers to treatment of cancer or tumors through the use of beam of ionizing radiation, as is well known in the art.

Radiotherapy uses high-energy x-rays given as external beam radiotherapy or internal beam radiotherapy to prevent or reduce further proliferation of cancer cells or to cause apoptosis in cancer cells. Although radiotherapy can affect both cancer cells as well as healthy cells, healthy cells are better able to resist or recover from the effects of radiation.

In one embodiment, a radiotherapy is administered in combination with the administration of an optional chemotherapy and/or an effective amount of a PD-1 antagonist.

In one embodiment, a radiotherapy may be administered before, during, or after a subject has started or ended a treatment regime comprising a chemotherapy and/or a therapeutically effective amount of a PD-1 antagonist.

In one embodiment, a radiotherapy is administered concurrently with a chemotherapy.

Radiotherapy is generally administered at a dose of about 1 Gy to about 200 Gy in one or more fractions.

In one embodiment, a radiotherapy is administered at a dose of about 10 Gy to about 150 Gy in one or more fractions.

In one embodiment, a radiotherapy is administered at a dose of about 20 Gy to about 100 Gy in one or more fractions.

In one embodiment, a radiotherapy is administered at a dose of about 20 Gy to about 80 Gy in one or more fractions.

In one embodiment, a radiotherapy is administered at a dose of about 60 Gy in one or more fractions.

In one embodiment, a radiotherapy is administered at a dose of about 60 Gy in 30 daily doses of 2 Gy.

### Standard Thoracic Radiotherapy

Prior to inclusion of any participant on this study, the radiation oncologist will evaluate the baseline thoracic computed tomography (CT) scan (preplanning CT simulation may be considered) in order to ensure that the tumor at baseline is anticipated to be treatable and treatment volumes are unlikely to significantly exceed the specified normal tissue constraints.

Participants in all groups will receive concurrent thoracic radiation therapy using a standardized 3D conformal radiation therapy (3DCRT) or Intensity-modulated radiation therapy (IMRT) technique on a linear accelerator operating at≥6 Mega-volt (MV) beam energy. 6 MV photons are preferred if possible; 10 MV photons may also be used. Use of photon energies higher than 10 MV is allowed but 6 to 10 MV energies are preferred. The target total dose of thoracic radiation therapy will be 60 Gy in 30 daily fractions of 2 Gy. There should be no planned breaks during the radiation except for national holidays and weekends. Proton treatment is not allowed.

4-Dimensional CT scan (4DCT) simulation is preferred. If a 4DCT scan simulation is not available, then a standard non-4DCT CT simulation is permitted with a motion management technique. Fluorodeoxyglucose (FD6)-positron emission tomography should be incorporated into treatment planning (i.e. image fusion).

Daily Image-guided radiation therapy (IGRT) using orthogonal X-ray, cone beam computed tomography (CBCT), CT on rails or magnetic resonance imaging (MM) guidance is used for all participants, regardless of the radiation technique. CBCT is preferred.

Participants will receive treatment 5 days per week, in once daily fractions, 2 Gy per fraction, to a target dose of 60 Gy in 30 fractions. The entire planning target volume (PTV) must be treated daily to 60 Gy. Resimulation to adjust for changes in tumor volumes may be performed when appropriate with replanning to achieve the dose constraints provided. Dose 2 of chemotherapy and pembrolizumab are given during the first week of thoracic radiation therapy. When both chemotherapy and radiation are administered at the same center/location, it is recommended that radiation should follow within 30 to 60 minutes of the completion of chemotherapy, especially on Day 1 of thoracic radiation therapy. When the radiotherapy is delivered at a separate location, logistic considerations may result in radiotherapy being delivered prior to the administration of chemotherapy. On days where thoracic radiation therapy and/or chemotherapy are delayed for administrative reasons (eg, holidays or weather), this will not be considered a protocol violation, provided the full planned dose of thoracic radiation therapy is administered.

### Chemotherapy

An optional chemotherapy is used in various methods and kits disclosed herein, An optional chemotherapy can be used in the treatment phase in combination with other treatments such as a radiotherapy and/or a PD-1 antagonist.

In some embodiments, a subject (e.g., a mammal, e.g., a human) for treatment with an PD-1 antagonist is treated with chemotherapy (e.g., platinum-based chemotherapy). In some embodiments, a chemotherapeutic agent is actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, vinorelbine, or a combination of any two or more of the forgoing chemotherapies.

In some such embodiments, a chemotherapeutic agent is a platinum based chemotherapeutic agent, such as cisplatin, carboplatin, oxaliplatin, nedaplatin, triplatin tetranitrate, phenanthriplatin, picoplatin, satraplatin or a combination of any two or more of the forgoing chemotherapies.

In one embodiment, the optional chemotherapy is administered and is selected from adriamycin, bleomycin, cisplatin, carboplatin, dactinomycin, daunorubicin, docetaxel, etoposide, irinotecan, mitomycin C, paclitaxel, pemetrexed, plicamycin, podophyllotoxin, topotecan, vincristine, and a combination of any two or more of the forgoing chemotherapies.

In one embodiment, the chemotherapy is selected from cisplatin, carboplatin, etoposide, paclitaxel, pemetrexed and a combination of any two of the foregoing chemotherapies.

In one embodiment, the chemotherapy is a platinum doublet selected from:
(1) a combination of cisplatin and pemetrexed;
(2) a combination of cisplatin and etoposide; and
(3) a combination of carboplatin and paclitaxel.

In one embodiment, the chemotherapy is a platinum doublet selected from:
(1) cisplatin 75 mg/m² IV and pemetrexed 500 mg/m² IV in three cycles (Day 1 in each cycle of Cycles 1-3);
(2) cisplatin 50 mg/m² IV (Days 1 and 8 in Cycles 1 and 2; Days 8 and 15 in Cycle 3) and etoposide 50 mg/m² IV in three cycles (Days 1 to 5 in Cycles 1 and 2; days 8 to 12 in Cycle 3); and
(3) carboplatin AUC 6 mg/mL/min IV with paclitaxel 200 mg/m² IV on Day 1 in Cycle 1; carboplatin AUC 2 mg/mL/min IV with paclitaxel 45 mg/m² IV on Days 1, 8, and 15 in Cycles 2 and 3.

In one embodiment, a chemotherapy is administered concurrently with a radiotherapy.

In one embodiment, prior to paclitaxel infusion, all participants should be premedicated with oral or intravenous corticosteroids, diphenhydramine, and H2 antagonists.

In one embodiment, prior to pemetrexed infusion, all participants should receive the appropriate supplementation of vitamin B12, folic acid and dexamethasone.

In one embodiment, all participants should receive the appropriate corticosteroid premedications as per the local approved label.

In one embodiment, additional premedications and pre- and post-cisplatin hydration should be administered as per standard practice.

Treatment with PARP Inhibitors

Poly(ADP-ribose) polymerase (PARP) enzymes are a family of enzymes that cleave NAD+, releasing nicotinamide, and successively add ADP-ribose units to form ADP-ribose polymers.

Accordingly, activation of PARP enzymes can lead to depletion of cellular NAD+ levels (e.g., PARPs as NAD+ consumers) and mediates cellular signaling through ADP-ribosylation of downstream targets. PARP-1 is a zinc-finger DNA-binding enzyme that is activated by binding to DNA double or single strand breaks. It was known that anti-alkylating agents could deplete the NAD+ content of tumor cells, and the discovery7 of PARPs explained this phenomenon. (PARP Inhibitors and Cancer Therapy. Curtin N. in Poly ADP Ribosylation. ed. Alexander Burke, Lands Bioscience and Springer Bioscience, 2006: 218-233). Anti-alkylating agents induce DNA strand breaks, which activates of PARP-1, which is part of the DNA repair pathway. Poly7 ADP-ribosylation of nuclear proteins by PARP-1 converts DNA damage into intracellular signals that can either activate DNA repair (e.g. by the base excision repair (BER) pathway); or trigger cell death in the presence of DNA damage that is too extensive and cannot be efficiently repaired.

PARP-2 contains a catalytic domain and is capable of catalyzing a poly(ADP-ribosyl)ation reaction. PARP-2 displays auto-modification properties similar to PARP-1. The protein is localized in the nucleus in vivo and may account for the residual poly(ADP-ribose) synthesis observed in PARP-1-deficient cells, treated with alkylating agents or hydrogen peroxide. Some agents that inhibit PARP (e.g., agents primarily aimed at inhibiting PARP-1) may also inhibit PARP-2 (e.g., niraparib).

The role of PARP enzymes in DNA damage response (e.g. repair of DNA in response to genotoxic stress) has led to the compelling suggestion that PARP inhibitors may be useful anti-cancer agents. PARP inhibitors may be particularly effective in treating cancers resulting from germ line or sporadic deficiency in the homologous recombination DNA repair pathway, such as BRCA-1 and/or BRCA-2 deficient cancers.

Pre-clinical ex vivo and in vivo experiments suggest that PARP inhibitors are selectively cytotoxic for tumors with homozygous inactivation of BRCA-1 and/or BRCA-2 genes, which are known to be important in the homologous recombination (HR) DNA repair pathway. The biological basis for the use of PARP inhibitors as single agents in cancers with defects in BRCA-1 and/or BRCA-2 is the requirement of PARP-1 and PARP-2 for base excision repair (BER) of the damaged DNA. Upon formation of single-strand DNA breaks, PARP-1 and PARP-2 bind at sites of lesions, become activated, and catalyze the addition of long polymers of ADP-ribose (PAR chains) on several proteins associated with chromatin, including histones. This results in chromatin relaxation and fast recruitment of DNA repair factors that access and repair DNA breaks. Normal cells repair up to 10,000 DNA defects daily and single strand breaks are the most common form of DNA damage. Celis with defects in the BER pathway enter S phase with unrepaired single strand breaks. Preexisting single strand breaks are converted to double strand breaks as the replication machinery passes through the break. Double strand breaks present during S phase are preferentially repaired by the error-free HR pathway. Cells with inactivation of genes required for HR, such as BRCA-1 and/or BRCA-2, accumulate stalled replication forks during S phase and may use error-prone non-homoiogous end joining (NHEJ) to repair damaged DNA. Both the inability to complete S phase (because of stalled replication forks) and error-prone repair by NHEJ, are thought to contribute to cell death.

Without wishing to be bound by theory, it is hypothesized that treatment with PARP inhibitors may selectively kill a subset of cancer cells with deficiencies in DNA repair pathways (e.g., inactivation of BRCA-1 and/or BRCA-2). For example, a tumor arising in a patient with a germline BRCA mutation has a defective homologous recombination DNA repair pathway and would be increasingly dependent on BER, a pathway blocked by PARP inhibitors, for maintenance of genomic integrity. This concept of inducing death by use of PARP inhibitors to block one DNA repair pathway in tumors with pre-existing deficiencies in a complementary DNA repair pathways is called synthetic lethality.

The therapeutic potential of PARP inhibitors is further expanded by the observation that PARP inhibitors not only have monotherapy activity in HR-deficient tumors, but are also effective in preclinical models in combination with other agents such as cisplatin, carboplatin, alkylating and methylating agents, radiation therapy, and topoisomerase I inhibitors. In contrast to the rationale for monotherapy in which PARP inhibition alone is sufficient for cell death in HR-deficient cancers (due to endogenous DNA damage), PARP is required for repair of DNA damage induced by standard cytotoxic chemotherapy. In some cases, the specific role of PARP is not known, but PARP is known to be required to release trapped topoisomerase I/irinotecan complexes from DNA. Temozolomide-induced DNA damage is repaired by the BER pathway, which requires PARP to recruit repair proteins. Combination therapies that enhance or synergize the cancer therapy without significantly increasing toxicity would provide substantial benefit to cancer patients, including ovarian cancer patients.

### Suitable PARP Inhibitors

Without wishing to be bound by theory, treatment with PARP inhibitors (e.g., PARP-1/2 inhibitors) as provided herein for the various methods and kits disclosed herein may selectively kill a subset of cancer cell types by exploiting their deficiencies in DNA repair. Human cancers exhibit genomic instability and an increased mutation rate due to underlying defects in DNA repair. These deficiencies render cancer cells more dependent on the remaining DNA repair pathways and targeting these pathways is expected to have an impact on the survival of the tumor cells than on normal cells.

In one embodiment, a PARP inhibitor is selected from ABT-767, AZD 2461, BGB-290, BGP 15, CEP 8983, CEP 9722, DR 2313, E7016, E7449, fluzoparib (SHR 3162), IMP 4297, IN01001, JPI 289, JPI 547, monoclonal antibody B3-LysPE40 conjugate, MP 124, niraparib (ZEJULA), NU 1025, NU 1064, NU 1076, NU1085, olaparib, 0N02231, PD 128763, R 503, R554, rucaparib (RUBRACA), SBP 101, SC 101914, Simmiparib, talazoparib (BMN-673), veliparib (ABT-888), and WW 46, 2-(4-(Trifluoromethyl)phenyl)-7,8-dihydro-5H-thiopyrano[4,3-d]pyrimidin-4-ol, or a pharmaceutically acceptable salt thereof.

In one embodiment, a PARP inhibitor is a small molecule. In one embodiment, a PARP inhibitor is an antibody agent. In one embodiment, an agent that inhibits PARP is a combination of agents.

In one embodiment, a PARP inhibitor is selected from olaparib, niraparib, rucaparib, talazoparib, veliparib, or any combination thereof. In one embodiment, a PARP inhibitor can be prepared as a pharmaceutically acceptable salt. In one embodiment, a salt form can exist as a solvated or hydrated polymorphic form.

In one embodiment, a PARP inhibitor is selected from olaparib, niraparib, rucaparib, and talazoparib, or a pharmaceutically acceptable salt thereof.

In one embodiment, the PARP inhibitor is olaparib, or a pharmaceutically acceptable salt thereof. In one embodiment, the PARP inhibitor is olaparib.

### Olaparib

Olaparib (AZD2281, KU-0059436) is a potent PARP inhibitor (PARP 1, 2, and 3) that is being developed as a monotherapy as well as for combination with chemotherapy, ionizing radiation and other anticancer agents including novel agents and immunotherapy. PARP inhibition is a novel approach to targeting tumors with deficiencies in DNA repair mechanisms. PARP enzymes are essential for repairing DNA single strand breaks (SSBs). Inhibiting PARPs leads to the persistence of SSBs, which are then converted to the more serious DNA double strand breaks (DSBs) during the process of DNA replication. During the process of cell division, DSBs can be efficiently repaired in normal cells by homologous recombinational repair (HRR). Tumors with homologous recombinational deficiency (HRD), such as ovarian cancers in patients with breast cancer susceptibility gene 1/2 (BRCA1/2) mutations, cannot accurately repair DNA damage, which may become lethal to cells as DNA abnormalities accumulate. In such tumor types, olaparib may offer a potentially efficacious and less toxic cancer treatment compared with currently available chemotherapy regimens. Olaparib traps the inactive form of PARP on DNA at sites of SSBs, thereby preventing their repair.

### Dosing and Administration

Further provided for the various methods and kits disclosed herein are dosing regimens and routes of administration for treating cancer (e.g., NSCLC) using a combination of a PD-1 antagonist (e.g., an anti-PD-1 monoclonal antibody or antigen binding fragment thereof), a radiotherapy, an optional chemotherapy, and a PARP inhibitor (e.g., olaparib or a pharmaceutically acceptable salt thereof).

The PD-1 antagonist (e.g., the anti-PD-1 monoclonal antibody or antigen binding fragment thereof), the radiotherapy, the optional chemotherapy or the poly(ADP-ribose) polymerase (PARP) inhibitor (e.g., olaparib or a pharmaceutically acceptable salt thereof) disclosed herein may be administered by doses administered, e.g., daily, 1-7 times per week, weekly, hi-weekly, every three weeks, every four weeks, every five weeks, every 6 weeks, monthly, bimonthly, quarterly, semiannually, annually, etc. Doses may be administered, e.g., intravenously, subcutaneously, topically, orally, nasally, rectally, intramuscular, intracerebrally, irtraspinally, or by inhalation. In certain embodiments, the doses are administered intravenously. In certain embodiments, the doses are administered subcutaneously. In certain embodiments, the doses are administered orally. A total dose for a treatment interval is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 4.0 mg/kg, 5.0 mg/ml, 10 mg/kg, 25 mg/kg, 50 mg/kg or more. Doses may also be provided to achieve a pre-determined target concentration of the antibody (e.g., anti-PD-1 antibody) or antigen binding fragment thereof in the subject's serum, such as 0.1, 0.3, 1, 3, 10, 30, 100, 300 μg/mL or more.

In some embodiments, the PD-1 antagonist anti-PD-1 monoclonal antibody or antigen binding fragment thereof) is administered subcutaneously or intravenously, on a weekly, biweekly, triweekly, every 4 weeks, every 5 weeks, every 6 weeks, monthly, bimonthly, or quarterly basis at 10, 20, 50, 80, 100, 200, 300, 400, 500, 1000 or 2500 mg/subject. In some specific methods, the dose of the PD-1 antagonist (e.g., anti-PD-1 monoclonal antibody or antigen binding fragment thereof) is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, or from about 2.0 mg/kg to about 4.0 mg/kg. In some specific methods, the dose of the PD-1 antagonist (e.g., anti-PD-1 monoclonal antibody or antigen binding fragment thereof) is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, or from about 200 mg to about 240 mg. In some embodiments, the dose of the PD-1 antagonist (e.g., anti-PD-1 monoclonal antibody or antigen binding fragment thereof) is 50 mg, 75 mg, 100 mg, 125 mg, 150 mg, 175 mg, 200 mg, 225 mg, 240 mg, 250 mg, 300 mg, 400 mg, or 500 mg.

In some embodiments of various methods described herein, the PD-1 antagonist is an anti-human PD-1 monoclonal antibody or antigen binding fragment thereof. In some embodiments, the anti-human PD-1 monoclonal antibody or antigen binding fragment thereof is pembrolizumab, the human patient is administered 200 mg, or 2 mg/kg pembrolizumab, and pembrolizumab is administered once every three weeks. In one embodiment, the human patient is administered 200 mg pembrolizumab once every three weeks. In one embodiment, the human patient is administered 2 mg/kg pembrolizumab once every three weeks.

In certain embodiments of various methods described herein, the anti-human PD-1 monoclonal antibody or antigen binding fragment thereof is pembrolizumab, the human patient is administered 400 mg pembrolizumab, and pembrolizumab is administered once every six weeks.

In other embodiments of various methods described herein, the anti-human PD-1 monoclonal antibody or antigen binding fragment thereof is nivolumab, the human patient is administered 240 mg or 3 mg/kg nivolumab, and nivolumab is administered once every two weeks. In one specific embodiment, the human patient is administered 240 mg nivolumab once every two weeks. In one specific embodiment, the human patient is administered 3 mg/kg nivolumab once every two weeks. In other embodiments of various methods described herein, the anti-human PD-1 monoclonal antibody or antigen binding fragment thereof is nivolumab, the human patient is administered 480 mg nivolumab, and nivolumab is administered once every four weeks.

In yet other embodiments of various methods described herein, the anti-human PD-1 monoclonal antibody or antigen binding fragment thereof is cemiplimab, the human patient is administered 350 mg cemiplimab, and cemiplimab is administered once every three weeks.

In some embodiments of the methods or kits provided herein, the PD-1 antagonist is an anti PD-L1 antibody or antigen binding fragment thereof.

In some embodiments of the methods or kits provided herein, the anti-PD-L1 antibody or antigen binding fragment thereof is avelumab, the human patient is administered 800 mg of avelumab, and aveulmab is administered once every two weeks.

In other embodiments embodiment of the methods or kits provided herein, the anti-PD-L1 antibody or antigen binding fragment thereof is atezolizumab, the human patient is administered 840 mg of atezolizumab, and the atezolizumab is administered once every two weeks. In some embodiments, the human patient is administered 1200 mg of atezolizumab, and the atezolizumab is administered once every three weeks. In yet other embodiments, the human patient is administered 1680 mg of atezolizumab, and the atezolizumab is administered once every four weeks.

In yet further embodiments of the methods or kits provided herein, the anti-PD-L1 antibody or antigen binding fragment thereof is durvalumab, the human patient is administered 10 mg/kg of durvalumab, and the durvalumab is administered once every two weeks. In one embodiment, the human patient is administered 1500 mg of durvalumab, and the durvalumab is administered once every three weeks. In another embodiment, the human patient is administered 1500 mg of durvalumab, and the durvalumab is administered once every four weeks.

In certain embodiments, a poly(ADP-ribose) polymerase (PARP) inhibitor (e.g., olaparib or a pharmaceutically acceptable salt thereof) is administered orally. In some embodiments, a poly(ADP-ribose) polymerase (PARP) inhibitor (e.g., olaparib or a pharmaceutically acceptable salt thereof) is administered at a daily dose of 100, 150, 200, 250, 300, 350, 400, 450, 500 or 550 mg each as olaparib. In certain embodiments, the PARP inhibitor is olaparib.

A total dose for a PARP inhibitor treatment as a part of the combination therapy disclosed herein is generally at least 10 mg, 20 mg, 30 mg, 40 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 rng, 800 mg, 850 mg, 900 mg, 950 mg or 1,000 mg twice daily.

In one embodiment, the PARP inhibitor is olaparib, or a pharmaceutically acceptable salt thereof, and is administered at a dose of 100 mg, 150 mg, 200 mg, 250 mg, 300 mg, 350 mg, 400 mg, 450 mg, 500 mg, 550 mg, 600 mg, 650 mg, 700 mg, 750 mg or 800 mg twice daily as a part of the combination therapy disclosed herein.

In another embodiment, the PARP inhibitor is olaparib, or a pharmaceutically acceptable salt thereof, and is administered at a dose of 200 mg, 250 mg, 300 mg, 350 mg, 400 mg, 450 mg, 500 mg, 550 mg or 600 mg twice daily as a part of the combination therapy disclosed herein.

In another embodiment, the PARP inhibitor is olaparib, or a pharmaceutically acceptable salt thereof, and is administered at a dose of 300 mg, 350 mg, 400 mg, 450 mg or 500 mg twice daily as a part of the combination therapy disclosed herein.

In another embodiment, the PARP inhibitor is olaparib, or a pharmaceutically acceptable salt thereof, and is administered at a dose of 350 mg, 400 mg or 450 mg twice daily as a part of the combination therapy disclosed herein.

In another embodiment, the PARP inhibitor is olaparib, or a pharmaceutically acceptable salt thereof, and is administered at a dose of 400 mg twice daily as a part of the combination therapy disclosed herein.

A total dose of about 1 Gy to about 200 Gy of a radiotherapy in one or more fractions per treatment cycle is generally administered as a part of the combination therapy disclosed herein. In one embodiment, 1 to 20 radiotherapy treatment cycles are administered. In one embodiment, 1 to 15 radiotherapy treatment cycles are administered. In one embodiment, 1 to 10 radiotherapy treatment cycles are administered. In one embodiment, 1 to 5 radiotherapy treatment cycles are administered. In one embodiment, 1 to 3 radiotherapy treatment cycles are administered. In one embodiment, 1 radiotherapy treatment cycle is administered.

In one embodiment, a total dose of about 1 Gy to about 150 Gy of a radiotherapy in one or more fractions per treatment cycle is administered as a part of the combination therapy for the methods and kits disclosed herein. In one embodiment, 1 to 15 radiotherapy treatment cycles are administered. In one embodiment, 1 to 10 radiotherapy treatment cycles are administered. In one embodiment, 1 to 5 radiotherapy treatment cycles are administered. In one embodiment, 1 to 3 radiotherapy treatment cycles are administered. In one embodiment, 1 radiotherapy treatment cycle is administered.

In one embodiment, a total dose of about 10 Gy to about 100 Gy of a radiotherapy in one or more fractions per treatment cycle is administered as a part of the combination therapy disclosed herein. In one embodiment, 1 to 10 radiotherapy treatment cycles are administered. In one embodiment, 1 to 5 radiotherapy treatment cycles are administered. In one embodiment, 1 to 3 radiotherapy treatment cycles are administered. In one embodiment, 1 to 2 radiotherapy treatment cycles are administered. In one embodiment, 1 radiotherapy treatment cycle is administered.

In one embodiment, a total dose of about 20 Gy to about 80 Gy of a radiotherapy in one or more fractions per treatment cycle is administered as a part of the combination therapy disclosed herein. In one embodiment, 1 to 5 radiotherapy treatment cycles are administered. In one embodiment, 1 to 4 radiotherapy treatment cycles are administered. In one embodiment, 1 to 3 radiotherapy treatment cycles are administered. In one embodiment, 1 to 2 radiotherapy treatment cycles are administered. In one embodiment, 1 radiotherapy treatment cycle is administered.

In one embodiment, a total dose of about 60 Gy of a radiotherapy in one or more fractions per treatment cycle is administered as a part of the combination therapy disclosed herein. In one embodiment, 1 to 5 radiotherapy treatment cycles are administered. In one embodiment, 1 to 4 radiotherapy treatment cycles are administered. In one embodiment, 1 to 3 radiotherapy treatment cycles are administered. In one embodiment, 1 to 2 radiotherapy treatment cycles are administered. In one embodiment, 1 radiotherapy treatment cycle is administered.

In one embodiment, a total dose of about 60 Gy of a radiotherapy in 30 daily doses of 2 Gy per treatment cycle is administered as a part of the combination therapy disclosed herein. In one embodiment, 1 to 5 radiotherapy treatment cycles are administered. In one embodiment, 1 to 4 radiotherapy treatment cycles are administered. In one embodiment, 1 to 3 radiotherapy treatment cycles are administered. In one embodiment, 1 to 2 radiotherapy treatment cycles are administered. In one embodiment, 1 radiotherapy treatment cycle is administered.

A total dose of 1-5000 mg/m² or 1-100 mg/mL/min AUC of each chemotherapy per treatment cycle may be administered as a part of the combination therapy for the methods and kits disclosed herein. In one embodiment, 1 to 15 chemotherapy treatment cycles are administered. In one embodiment, 1 to 10 chemotherapy treatment cycles are administered. In one embodiment, 1 to 3 radiotherapy treatment cycles are administered. In one embodiment, 1 to 2 chemotherapy treatment cycles are administered. In one embodiment, 1 chemotherapy treatment cycle is administered.

In one embodiment, a total dose of 5-2000 mg/m² of each chemotherapy per treatment cycle is administered as a part of the combination therapy disclosed herein. In one embodiment, 1 to 15 chemotherapy treatment cycles are administered. In one embodiment, 1 to 10 chemotherapy treatment cycles are administered. In one embodiment, 1 to 3 radiotherapy treatment cycles are administered. In one embodiment, 1 to 2 chemotherapy treatment cycles are administered. In one embodiment, 1 chemotherapy treatment cycle is administered.

In one embodiment, a total dose of 10-1000 mg/m² of each chemotherapy per treatment cycle is administered as a part of the combination therapy disclosed herein. In one embodiment, 1 to 10 chemotherapy treatment cycles are administered. In one embodiment, 1 to 5 chemotherapy treatment cycles are administered. In one embodiment, 1 to 3 radiotherapy treatment cycles are administered. In one embodiment, 1 to 2 chemotherapy treatment cycles are administered. In one embodiment, 1 chemotherapy treatment cycle is administered.

In one embodiment, a total dose of 10-500 mg/m² of each chemotherapy per treatment cycle is administered as a part of the combination therapy disclosed herein. In one embodiment, 1 to 10 chemotherapy treatment cycles are administered. In one embodiment, 1 to 5 chemotherapy treatment cycles are administered. In one embodiment, 1 to 3 radiotherapy treatment cycles are administered. In one embodiment, 1 to 2 chemotherapy treatment cycles are administered. In one embodiment, 1 chemotherapy treatment cycle is administered.

In one embodiment, a total dose of 1-50 mg/mL/min AUC of each chemotherapy per treatment cycle is administered as a part of the combination therapy disclosed herein. In one embodiment, 1 to 5 chemotherapy treatment cycles are administered. In one embodiment, 1 to 3 radiotherapy treatment cycles are administered. In one embodiment, 1 to 2 chemotherapy treatment cycles are administered. In one embodiment, 1 chemotherapy treatment cycle is administered.

In one embodiment, a total dose of 1-10 mg/mL/min AUC of each chemotherapy per treatment cycle is administered as a part of the combination therapy disclosed herein. In one embodiment, 1 to 5 chemotherapy treatment cycles are administered. In one embodiment, 1 to 3 radiotherapy treatment cycles are administered. In one embodiment, 1 to 2 chemotherapy treatment cycles are administered. In one embodiment, 1 chemotherapy treatment cycle is administered.

In one embodiment, a total dose of 5-10 mg/mL/min AUC of each chemotherapy per treatment cycle is administered as a part of the combination therapy disclosed herein. In one embodiment, 1 to 5 chemotherapy treatment cycles are administered. In one embodiment, 1 to 3 radiotherapy treatment cycles are administered. In one embodiment, 1 to 2 chemotherapy treatment cycles are administered. In one embodiment, 1 chemotherapy treatment cycle is administered.

In one embodiment, a combination chemotherapy selected from the following is administered:
(1) cisplatin 75 mg/m² IV and pemetrexed 500 mg/m² IV in three cycles (Day 1 in each cycle of Cycles 1-3);
(2) cisplatin 50 mg/m² IV (Days 1 and 8 in Cycles 1 and 2; Days 8 and 15 in Cycle 3) and etoposide 50 mg/m² IV in three cycles (Days 1 to 5 in Cycles 1 and 2; days 8 to 12 in Cycle 3); and
(3) carboplatin AUC 6 mg/mL/min IV with paclitaxel 200 mg/m² IV on Day 1 in Cycle 1; carboplatin AUC 2 mg/mL/min IV with paclitaxel 45 mg/m² IV on Days 1, 8, and 15 in Cycles 2 and 3.

### Pharmaceutical Kits

In one embodiment, provided herein are pharmaceutical kits comprising the therapeutic agents disclosed herein (e.g., a PD-1 antagonist, a radiotherapy, a chemotherapy, and olaparib, or pharmaceutical compositions thereof), packaged into suitable packaging material. A kit optionally includes a label or packaging insert that include a description of the components or instructions for use in vitro, in vivo, or ex vivo, of the components therein.

In one embodiment, a pharmaceutical kit comprises:
(a) a PD-1 antagonist;
(b) instructions on administering a radiotherapy;
(c) a PARP inhibitor; and
(d) optionally, a chemotherapy.

In one embodiment, the PD-1 antagonist is an anti-PD-1 antibody. In another embodiment, the PD-1 antagonist is an anti-PD-L1 antibody.

In one embodiment, a pharmaceutical kit comprises:
(a) an anti-PD-1 antibody selected from pembrolizumab, nivolumab and cemiplimab;
(b) instructions on administering a radiotherapy as part of a treatment phase; (c) a PARP inhibitor selected from olaparib, niraparib, rucaparib, and talazoparib, or a pharmaceutically acceptable salt thereof; and
(d) a chemotherapy selected from cisplatin, carboplatin, etoposide, paclitaxel, pemetrexed and a combination of any two of the foregoing chemotherapies.

In one embodiment, a pharmaceutical kit comprises:
(a) an anti-PD-L1 antibody selected from atezolizumab, durvalumab and avelumab;
(b) instructions on administering a radiotherapy as part of a treatment phase;
(c) a PARP inhibitor selected from olaparib, niraparib, rucaparib, and talazoparib, or a pharmaceutically acceptable salt thereof; and
(d) a chemotherapy selected from cisplatin, carboplatin, etoposide, paclitaxel, pemetrexed and a combination of any two of the foregoing chemotherapies.

In one embodiment, a pharmaceutical kit comprises:
(a) an anti-PD1 antibody which is pembrolizumab;
(b) instructions on administering a radiotherapy at a dose of about 20 Gy to about 80 Gy as part of a treatment phase;
(c) a PARP inhibitor which is olaparib, or a pharmaceutically acceptable salt thereof; and
(d) a chemotherapy selected from:
   (1) a combination of cisplatin and pemetrexed;
   (2) a combination of cisplatin and etoposide; and
   (3) a combination of carboplatin and paclitaxel.

In one embodiment, the pharmaceutical kit described above further comprises instructions for administering to a human patient (a) the PD-1 antagonist, (b) the radiotherapy, (c) the PARP inhibitor, and optionally, (d) the chemotherapy.

In one embodiment, PD-1 antagonist is an anti-PD1 monoclonal antibody or antigen binding fragment thereof. In one embodiment, the anti-PD-1 monoclonal antibody or antigen binding fragment thereof is pembrolizumab. In one embodiment, the anti-PD-1 monoclonal antibody or antigen binding fragment thereof is nivolumab. In one embodiment, the anti-PD-1 monoclonal antibody or antigen binding fragment thereof is cemiplimab.

In one embodiment, the PARP inhibitor is olaparib, or a pharmaceutically acceptable salt thereof. In one embodiments, the PARP inhibitor is niraparib, or a pharmaceutically acceptable salt thereof.

The dosages for the PD-1 antagonist (e.g, an anti-PD-1 monoclonal antibody), the radiotherapy, the chemotherapy, or the PARP inhibitor described above can be used in various kits herein.

In one embodiment, a kit comprises dosages of each component sufficient for a certain period of treatment (e.g., 1, 2, 3, 4, 5, 6, 12, 24, 36, 48, 52 weeks, etc.). For example, a kit can comprise a dosage of 200 mg pembrolizumab, a dosage of chemotherapy for 3 cycles of treatment, which are sufficient for a 3-week treatment phase.

In some embodiments, the kit comprises means for separately retaining the components, such as a container, divided bottle, or divided foil packet. A kit of this disclosure can be used for administration of different dosage forms, for example, oral and parenteral, for administration of the separate compositions at different dosage intervals, or for titration of the separate compositions against one another.

### Uses of a Therapeutic Combination for Treating Cancer

In one embodiment, provided herein are uses of a therapeutic combination for treating cancer (e.g., NSCLC) in a human patient, wherein the therapeutic combination comprises:
(a) an effective amount of one or more programmed cell death 1 (PD-1) or programmed cell death ligand 1 (PD-L1) antagonists,
(b) an effective amount of a radiotherapy,
(c) an effective amount of a PARP inhibitor, and
optionally, (d) an effective amount of one or more chemotherapies.

These uses may be employed for the various methods and kits disclosed herein.

In one embodiment, a use of a therapeutic combination for treating cancer in a human patient comprises administering (a) a PD-1 antagonist, (b) a radiotherapy, (c) a PARP inhibitor, and (d) a chemotherapy, according to the following:
(1) a treatment phase comprising administering an effective amount of a PD-1 antagonist in combination with an effective amount of a radiotherapy and an effective amount of a chemotherapy; and followed by
(2) a maintenance phase comprising administering an effective amount of a PD-1 antagonist in combination with an effective amount of a PARP inhibitor.

In one embodiment, a use of a therapeutic combination for treating cancer in a human patient comprises administering:
(1) a treatment phase comprising administering an effective amount of a PD-1 antagonist in combination with an effective amount of a radiotherapy and an effective amount of a chemotherapy;
wherein the PD-1 antagonist is administered once or multiple times;
wherein the radiotherapy is administered at a dose of about 20 Gy to about 80 Gy in one or more fractions; and
wherein the chemotherapy is administered once or multiple times; and followed by
(2) a maintenance phase comprising administering an effective amount of a PD-1 antagonist in combination with an effective amount of a PARP inhibitor;
wherein the PD-1 antagonist is administered once or multiple times up to 12 months; and
wherein the PARP inhibitor is administered once or multiple times up to 12 months.

In one embodiment, a use of a therapeutic combination for treating cancer in a human patient comprises:
(1) a treatment phase comprising administering a PD-1 antagonist in combination with the radiotherapy and a chemotherapy;
wherein the PD-1 antagonist is pembrolizumab administered at a dose of 200 mg once every three weeks;
wherein the radiotherapy is a standard thoracic radiotherapy administered at a dose of about 60 Gy in fractions of 30 daily doses of 2 Gy each;
wherein the chemotherapy is selected from cisplatin, carboplatin, etoposide, paclitaxel, pemetrexed and a combination of any two of the foregoing chemotherapies; and
administered at a dose of 10-2000 mg/m² of each chemotherapy up to 3 cycles; and followed by
(2) a maintenance phase comprising administering a PD-1 antagonist in combination with a PARP inhibitor;
wherein the PD-1 antagonist pembrolizumab is administered at a dose of 200 mg once every three weeks for up to 12 months; and
wherein the PARP inhibitor is olaparib, or a pharmaceutically acceptable salt thereof, administered at a dose of 300 mg twice daily for up to 12 months.

In one embodiment, a use of a therapeutic combination for treating cancer in a human patient comprises:
(1) a treatment phase comprising administering a PD-1 antagonist in combination with the radiotherapy and a chemotherapy;
wherein the PD-1 antagonist is pembrolizumab administered at a dose of 400 mg once every six weeks;
wherein the radiotherapy is a standard thoracic radiotherapy administered at a dose of about 60 Gy in fractions of 30 daily doses of 2 Gy each;
wherein the chemotherapy is selected from cisplatin, carboplatin, etoposide, paclitaxel, pemetrexed and a combination of any two of the foregoing chemotherapies; and
administered at a dose of 10-2000 mg/m² of each chemotherapy up to 3 cycles; and followed by
(2) a maintenance phase comprising administering a PD-1 antagonist in combination with a PARP inhibitor;
wherein the PD-1 antagonist pembrolizumab is administered at a dose of 400 mg once every six weeks for up to 12 months; and
wherein the PARP inhibitor is olaparib, or a pharmaceutically acceptable salt thereof, administered at a dose of 300 mg twice daily for up to 12 months.

In one embodiment of the method, kit or use disclosed herein, the cancer is selected from the group consisting of bladder cancer, breast cancer, colorectal cancer, hepatocellular carcinoma, melanoma, non-small cell lung cancer (NSCLC), ovarian cancer, pancreatic cancer, prostate cancer, and renal cell carcinoma.

In one embodiment, the cancer is NSCLC.

In one embodiment, the cancer is unresectable, locally advanced, Stage III NSCLC.

Further disclosed herein is the use of any combination or therapeutic combination disclosed herein that comprises:
(a) an effective amount of one or more programmed death 1 (PD-1) antagonists;
(b) an effective amount of a radiotherapy;
(c) an effective amount of one or more poly(ADP-ribose) polymerase (PARP) inhibitors; and
(d) optionally, an effective amount of one or more chemotherapies;
or a combination of the foregoing for the manufacture of a medicament for the treatment of cancer.

In one embodiment, the present invention provides a combination or therapeutic combination as disclosed herein that comprises:
(a) an effective amount of one or more programmed death 1 (PD-1) antagonists;
(b) an effective amount of a radiotherapy;
(c) an effective amount of one or more poly(ADP-ribose) polymerase (PARP) inhibitors; and
(d) optionally, an effective amount of one or more chemotherapies;
or a combination of the foregoing for use in the treatment of cancer.

A number of embodiments of the invention have been described. It will be understood that various modifications may be made without departing from the spirit and scope of the invention. It will be further understood that each embodiment may be combined with one or more other embodiments, to the extent that such a combination is consistent with the description of the embodiments.

### EXAMPLES

The examples disclosed herein are offered by way of illustration, and not by way of limitation.

### Example 1: Phase 2 Clinical Trial of Pembrolizumab in Combination with Platinum Doublet Chemotherapy and Radiotherapy in Participatents with Unresectable, Locally Advanced Stage III Non-Small Cell Lung Cancer (NSCLC)

Concurrent platinum doublet chemotherapy with radiotherapy (CCRT) is a standard of care for patients with unresectable stage III NSCLC; however, CCRT does not reduce the risk of distant relapse and provide a low 5-year survival rate.

The table below provides baseline characteristics of all treated patients (with data cutoff date of Jan. 3, 2020).

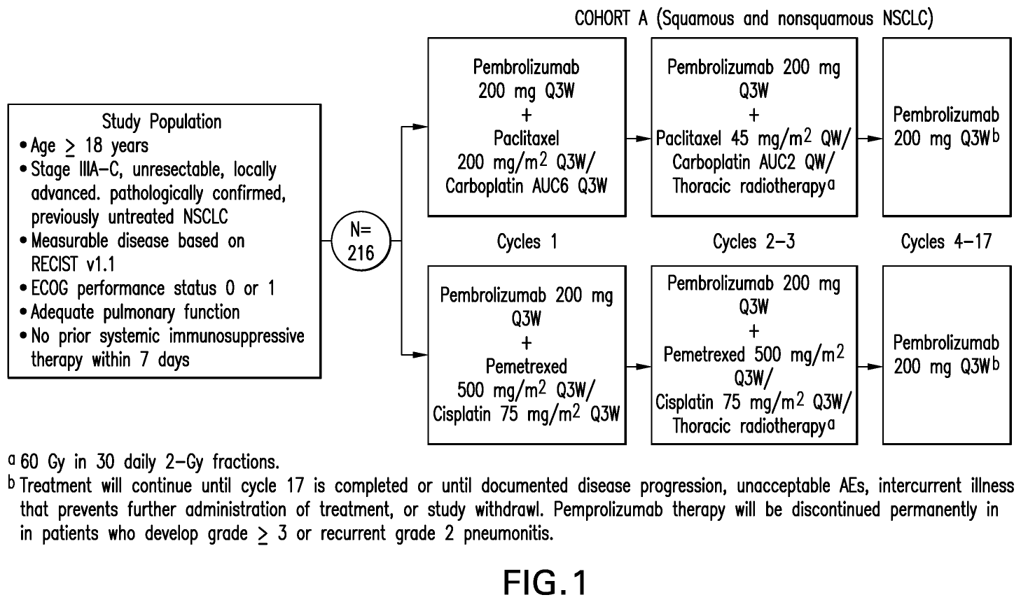

This is a non-randomized, open label study. FIG. 1 sets forth a schematic of the study. The primary objective is overall response rate (ORR) per response evaluation criteria in solid tumors (RECIST) version 1.1 by blinded independent central review (BICR). Secondary objectives are progression free survival (PFS), overall survival (OS) and safety. Key eligibility criteria are as set forth in FIG. 1 and include Stage IIIA-C, unresectable, locally advanced, pathologically confirmed, previously untreated NSCLC; measurable disease based on RECIST v1.1; eastern cooperative oncology group (ECOG) performance status 0 or 1; adequate pulmonary function; and no prior systemic immunosuppressive therapy within 7 days.

| N = number | Cohort A; N = 112 | | Cohort B; N = 73 | |
|---|---|---|---|---|
| Age, median (range), year | 66.0 | (46-90) | 64.0 | (35-78) |
| Men, n (%) | 76 | (67.9) | 40 | (54.8) |
| ECOG PS 1, n (%) | 61 | (54.5) | 34 | (46.6) |
| Squamous, n (%) | 73 | (65.2) | 0 | |
| Nonsquamous, n (%) | 39 | (34.8) | 73 | (100) |
| Former/current smoker, n (%) | 106 | (94.6) | 70 | (95.9) |
| PD-L1 Tumor Propotion Score | 66 | (58.9) | 30 | (41.1) |

(TPS) ≥ 1%

The table below provides ORR and Duration of Response/Efficacy by BICR per RECIST v1.1, patients with ≥15 weeks follow up (Jan. 3, 2020 data cutoff date)

| | Cohort A; N = 112 | | Cohort B; N = 53 | |
|---|---|---|---|---|
| ORR, n (%) [90% CI (confidence | 75 | (67.0) [58.9-74.3] | 30 | (56.6) [44.4-68.2] |
| interval)] | | | | |
| Complete response (CR) | 3 | (2.7) | 2 | (3.8) |
| Partial response (PR) | 72 | (64.3) | 28 | (52.8) |
| Standard deviation (SD), n (%) | 23 | (20.5) | 18 | (34.0) |
| PD, n (%) | 1 | (0.9) | 0 | |
| Not evaluable, n (%) | 3 | (2.7) | 0 | |
| No Assessment, n (%) | 10 | (8.9) | 5 | (9.4) |
| Duration of response, median (range)^{a}, mo | NR (1.6+ to 10.5+) | | NR (1.7+ to 10.5+) | |
| Response duration ≥6 month^{a}, n (%) | 30 | (91.1) | 9 | (100) |

| | | |
|---|---|---|
| 6-mo PFS rate^{a}, % | 81.4 | 85.2 |
| 6-mo OS rate^{a}, % | 87.2 | 94.8 |

^{a}Kaplan-Meier estimate. “+” indicates there is no progressive disease by the time of last disease assessment. Data cutoff date: Jan. 3, 2020.

PFS and OS were assessed in patients with at least 15 weeks follow up. As shown in the table above, at the data cutoff, few patients in either cohort had experienced disease progression or died. The progression free survival rate at 6 months was greater than 80% in both cohorts and the overall survival rate at 6 months was 87% in cohort A and 95% in cohort B. Median progression free survival and overall survival had not been reached in either cohort.

A secondary primary objective of the study was incidence of grade≥3 pneumonitis, which was assessed in all enrolled patients, regardless of their follow-up duration (see table below). In both cohorts, the rate of grade≥3 pneumonitis was less than 10%. One patient in cohort B had interstitial lung disease; the remaining patients had either pneumonitis or radiation pneumonitis. Incidence of other adverse events was consistent with that reported in other pembrolizumab monotherapy studies in NSCLC. Immune-mediated adverse events and infusion reactions (regardless of attribution to study treatment by the investigator) occurred in 47% of patients in cohort A and 27% in cohort B. 15% of patients in cohort A and 8% in cohort B experienced grade 2-5 immune mediated adverse events or infusion reactions.

| | Cohort A; N = 112 | | Cohort B; N = 73 | |
|---|---|---|---|---|
| Grade ≥3 pneumonitis (all cause),^{a} | 9 | (8.0) [4.3-13.6] | 4 | (5.5) [1.9-12.1] |
| n(5) [90% CI] | | | | |
| Treatment Releated Adverse Events | 105 | (93.8) | 64 | (87.7) |
| Grade 3-5 | 72 | (64.3) | 30 | (41.1) |
| Led to death | 4^{a} | (3.6) | 0 | |
| Led to discontinuation of any | 32 | (28.6) | 9 | (12.3) |
| treatment component | | | | |
| Immune-mediated adverse events | 53 | (47.3) | 20 | (27.4) |
| and infusion reactions | | | | |
| Grades 3-5 | 17 | (15.2) | 6 | (8.2) |

| | | | |
|---|---|---|---|
| Led to death | 4 | (3.6) | 0 |

^{a}Four (3.6%) of patients in cohort A and none in cohort B had treatment-related grade 5 pneumonitis. Data cutoff date: Jan. 3, 2020

As can be seen from above, pembrolizumab plus CCRT shows promising antitumor activity in patients with unresectable, locally advanced stage III NSCLC. ORR in both cohorts exceeded 50%. Estimated response duration was≥6 months for most patients with a response. The incidence of adverse events among patients who received pembrolizumab pluse CCRT was consistent with the established toxicity profiles of CCRT for stage III NSCLC and pembrolizumab monotherapy (See, e.g., Yoon, SM World J Clin Oncol 2017; 8-20 and Mok T., et al., Lancet 2019; 393:1819-1830). Observed rates of grade≥3 pneumonitis were within the expected range for immunotherapy combined with CCRT (See, e.g., Peters S., et al., Lung Cancer, 2019; 133:83-87).

### Example 2: Phase 3 Clinical Trial of Administering an Anti-PD-1 Antibody in Combination with a Radiotherapy and Chemotherapy Followed by Administering an Anti-PD-1 Antibody and Olaparib in NSCLC Patients

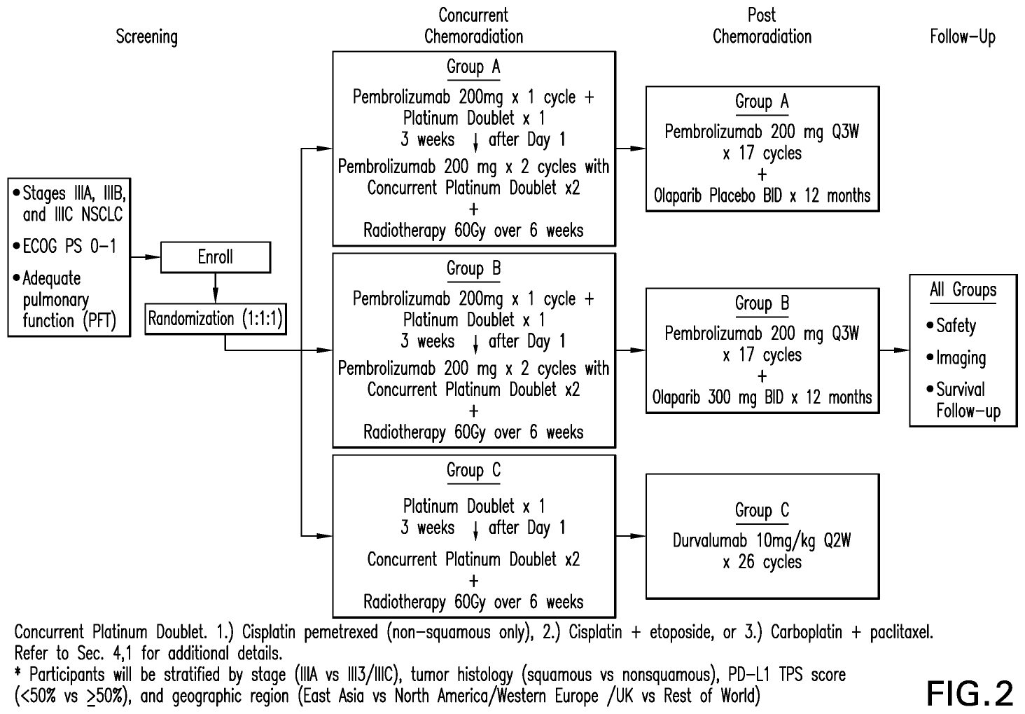

The purpose of this study is to assess the efficacy and safety of pembrolizumab in combination with concurrent chemoradiation therapy followed by either pembrolizumab with olaparib placebo (Arm 1) or with olaparib (Arm 2) compared to concurrent chemoradiation followed by durvalumab (Arm 3) in patients with unresectable, locally advanced non-small cell lung cancer (NSCLC). Arms 1 and 2 will be studied in a double-blind design and Arm 3 will be open label. A schematic of the clinical trial is set forth in FIG. 2.

Primary Outcome Measures includes Progression-Free Survival (PFS) up to approximately 48 months according to Response Evaluation Criteria in Solid Tumors Version 1.1 (RECIST 1.1) as assessed by blinded independent central review (BICR). PFS is defined as the time from randomization to the first documented disease progression or death due to any cause, whichever occurs first. Overall Survival (OS), up to approximately 72 months) is also a primary outcome measure. OS is the time from randomization to death due to any cause.

Secondary Outcome Measures are as set forth in the table below:

| Outcome Measure | Time Frame | Description |
|---|---|---|
| Incidence of Adverse | Up to | An AE is defined as any untoward medical |
| Events (AE) | approximately | occurrence in a clinical study participant, |
| | 72 months | temporally associated with the use of study |
| | | intervention, whether or not considered related |
| | | to the study intervention. |
| Discontinuation Rate of | Up to | An AE is defined as any untoward medical |
| Study Intervention Due to | approximately | occurrence in a clinical study participant, |
| an Adverse Event (AE) | 72 months | temporally associated with the use of study |
| | | intervention, whether or not considered related |
| | | to the study intervention. |
| Objective Response Rate | Up to | ORR is defined as the percentage of |
| (ORR) Per Response | approximately | participants who have achieved a Complete |
| Evaluation Criteria in Solid | 72 months | Response (CR) or a Partial Response (PR). |
| Tumors Version 1.1 | | |
| (RECIST 1.1) as Assessed | | |
| by Blinded Independent | | |
| Central Review (BICR | | |
| Duration of Response | Up to | DOR is defined as the time from first |
| (DOR) Per Response | approximately | documented evidence of Complete Response |
| Evaluation Criteria in Solid | 72 months | (CR) or a Partial Response (PR) until disease |
| Tumors Version 1.1 | | progression or death due to any cause, |
| (RECIST 1.1) as Assessed | | whichever occurs first. |
| by Blinded Independent | | |
| Central Review (BICR) | | |
| Change from Baseline in | Baseline (at | The EORTC QLQ-C30 is a questionnaire to |
| EORTC Quality of Life | randomization) | assess the overall quality of life of cancer |
| Questionnaire-Core 30 | and at the end | patients. Participant responses to the questions |
| (QLQ-C30) Global Health | of study | “How would you rate your overall health during |
| Status/Quality of Life | (approximately | the past week?” and “How would you rate your |
| (Items 29 and 30) Scale | 72 months post | overall quality of life during the past week?” |
| Score | randomization) | are scored on a 7-point scale (1 = Very poor to |
| | | 7 = Excellent). Using linear transformation, raw |
| | | scores are standardized, so that scores range |
| | | from 0 to 100. A higher score indicates a better |
| | | overall health status. The change from baseline |
| | | in EORTC QLQ-C30 Items 29 and 30 scale |
| | | scores will be presented. |
| Change From Baseline 1 in | Baseline (at | The EORTC QLQ-LC13 is a supplemental lung |
| Cough Using the European | randomization) | cancer-specific questionnaire that includes a |
| Organization for Research | and at the end | single-item scale score for cough (Item 1). For |
| and Treatment of Cancer | of study | this item, individual responses to the question |
| Quality of Life | (approximately | “How much did you cough?” are given on a 4- |
| Questionnaire Lung Cancer | 72 months post | point scale (1 = Not at all; 4 = Very much). Using |
| Module 13 | randomization) | linear transformation, raw scores are |
| (EORTC QLQ-LC13) Item | | standardized, so that scores range from 0 to |
| 1 Score | | 100, with a lower score indicating a better |
| | | outcome. The change from baseline in the |
| | | EORTC QLQ-LC13 cough scale score will be |
| | | presented. |
| Change From Baseline 1 in | Baseline (at | The EORTC QLQ-LC13 is a supplemental lung |
| Chest Pain Using the | randomization) | cancer-specific questionnaire that includes a |
| EORTC QLQ-LC13 Item | and at the end | single-item scale score for chest pain (Item 10). |
| 10 Score | of study | For this item, individual responses to the |
| | (approximately | question “Have you had pain in your chest?” |
| | 72 months post | are given on a 4-point scale (1 = Not at all; |
| | randomization) | 4 = Very much). Using linear transformation, |
| | | raw scores are standardized, so that scores |
| | | range from 0 to 100, with a lower score |
| | | indicating a better outcome. The change from |
| | | baseline in the EORTC QLQ-LC13 chest pain |
| | | scale score will be presented. |
| Change From Baseline in | Baseline (at | The EORTC QLQ-C30 is a questionnaire to |
| Dyspnea Using the EORTC | randomization) | assess the overall quality of life of cancer |
| QLQ-C30 Item 8 Score | and at the end | patients and includes a single-item scale score |
| | of study | for dyspnea (Item 8). Participant responses to |
| | (approximately | the question |
| | 72 months post | “Were you short of breath? are scored on a 4- |
| | randomization) | point scale (1 = not at all to 4 = very much). Using |
| | | linear transformation, raw scores are |
| | | standardized, so that scores range from 0 to |
| | | 100, with a lower score indicating a better |
| | | outcome. The change from baseline in the |
| | | EORTC QLQ-C30 dyspnea scale score will be |
| | | presented. |
| Change From Baseline in | Baseline (at | The EORTC QLQ-C30 is a questionnaire to |
| Physical Functioning Using | randomization) | assess the overall quality of life of cancer |
| the EORTC QLQ-C30 | and at the end | patients. The physical functioning scale consists |
| Items 1-5 Score | of study | of participant responses to 5 questions |
| | (approximately | regarding performance of daily activities [1) |
| | 72 months post | strenuous activities; 2) long walks; 3) short |
| | randomization) | walks; 4) bed/chair rest; and 5) needing help |
| | | with eating, dressing, washing themselves or |
| | | using the toilet]. Participant responses are |
| | | scored on a 4-point scale (1 = Not at All to |
| | | 4 = Very Much). Using linear transformation, |
| | | raw scores are standardized, so that scores |
| | | range from 0 to 100, with a higher score |
| | | indicating a better quality of life. The change |
| | | from baseline in the EORTC QLQ-C30 |
| | | physical functioning scale score will be |
| | | presented. |
| Time to Deterioration | Up to | TTD is defined as the time to first onset of a ≥10- |
| (TTD) in HRQoL Using the | approximately | point decrease from baseline for EORTC |
| EORTC QLQ-C30 Items 29 | 72 months post | QLQ-C30 Items 29 and 30 scale scores. |
| and 30 Score | randomization | |
| TTD in Cough Using the | Up to | TTD is defined as the time to first onset of a ≥10- |
| EORTC QLQ-LC13 Item 1 | approximately | point decrease from baseline for EORTC |
| Score | 72 months post | QLQ-LC13 Item 1 scale score. |
| | randomization | |
| TTD in Chest Pain Using | Up to | TTD is defined as the time to first onset of a ≥10- |
| the EORTC QLQ-LC13 | approximately | point decrease from baseline for EORTC |
| Item 10 Score | 72 months post | QLQ-LC13 Item 10 scale score. |
| | randomization | |
| TTD in Dyspnea Using the | Up to | TTD is defined as the time to first onset of a ≥10- |
| EORTC QLQ-C30 Item 8 | approximately | point decrease from baseline for EORTC |
| Score | 72 months post | QLQ-C30 Item 8 scale score. |
| | randomization | |
| TTD in Physical | Up to | TTD is defined as the time to first onset of a ≥10- |
| Functioning Using the | approximately | point decrease from baseline for EORTC |
| EORTC QLQ-C30 Items | 72 months post | QLQ-C30 Items 1-5 scale scores. |
| 1-5 Score | randomization | |

### Study Design

All participants will be randomized (1:1:1) into 3 study groups (Groups A, B, and C) and receive the following interventions:

### Group A

Participants will receive pembrolizumab 200 mg IV Q3W in combination with 3 cycles of platinum doublet chemotherapy and concurrent standard thoracic radiotherapy (60 Gy in 2 Gy fractions; during Cycles 2 and 3) followed by pembrolizumab plus matching olaparib placebo for 12 months or until specific discontinuation criteria are met.

### Group B

Participants will receive pembrolizumab 200 mg IV Q3W in combination with 3 cycles of platinum doublet chemotherapy and concurrent standard thoracic radiotherapy (60 Gy in 2 Gy fractions; during Cycles 2 and 3) followed by pembrolizumab plus olaparib (300 mg BID) for 12 months or until specific discontinuation criteria are met.

### Group C

Participants will receive 3 cycles of platinum doublet chemotherapy and concurrent standard thoracic radiotherapy (60 Gy in 2 Gy fractions; during Cycles 2 and 3) followed by durvalumab 10 mg/kg Q2W for 12 months or until specific discontinuation criteria are met.

Platinum doublet options (per investigator choice) allowed in the study include:
Cisplatin 75 mg/m² IV and pemetrexed 500 mg/m² IV Q3W (Day 1 in each cycle of Cycles 1-3) (nonsquamous histology only).
Cisplatin 50 mg/m² IV (Days 1 and 8 in Cycles 1 and 2; Days 8 and 15 in Cycle 3); etoposide 50 mg/m² IV (Days 1 to 5 in Cycles 1 and 2; Days 8 to 12 in Cycle 3).
Carboplatin AUC 6 mg/mL/min IV with paclitaxel 200 mg/m² IV on Day 1 in Cycle 1; carboplatin AUC 2 mg/mL/min IV with paclitaxel 45 mg/m² IV on Days 1, 8, and 15 in Cycles 2 and 3.

### Inclusion Criteria

Participants are eligible to be included in the study only if all of the following criteria apply:
has pathologically (histologically or cytologically) confirmed NSCLC;
has Stage IIIA, IIIB, or IIIC NSCLC by American Joint Committee on Cancer Version 8;
is unable to undergo surgery with curative intent for Stage III NSCLC;
has no evidence of metastatic disease indicating Stage IV NSCLC;
has measurable disease as defined by RECIST 1.1;
has not received prior treatment (chemotherapy, targeted therapy or radiotherapy) for Stage III NSCLC;
has provided a tumor tissue sample (tissue biopsy [core, incisional, or excisional]);
has an Eastern Cooperative Oncology Group (ECOG) Performance Status (PS) of 0 or 1 assessed within 7 days prior to the first administration of study intervention;
has a life expectancy of at least 6 months;
a male participant must agree to use contraception and refrain from donating sperm during the treatment period and for at least 180 days following the last dose of study treatment;
a female participant is eligible to participate if she is not pregnant, not breastfeeding, and agrees to use contraception during the treatment period and for at least 180 days following the last dose of study treatment;
has adequate pulmonary function tests;
has adequate organ function; and
has provided written informed consent.

### Exclusion Criteria:

Participants are excluded from the study if any of the following criteria apply:
has small cell lung cancer or a mixed tumor with presence of small cell elements;
has history, current diagnosis, or features suggestive of myelodysplastic syndrome (MDS)/acute myeloid leukemia (AML);
has had documented weight loss>10% (from baseline) in the preceding 3 months;
has received prior radiotherapy to the thorax, including radiotherapy to the esophagus, mediastinum, or for breast cancer;
has received prior therapy with an anti-programmed cell death 1 (ant-PD-1), anti-programmed cell death ligand 1 (anti-PD-L1), or anti-programmed cell death ligand 2 (anti-PD-L2) agent or with an agent directed to another stimulatory or co-inhibitory T-cell receptor;
has received prior therapy with olaparib or with any other polyadenosine 5′ diphosphoribose (polyADP ribose) polymerization (PARP) inhibitor;
has had major surgery<4 weeks prior to the first dose of study treatment (except for placement of vascular access);
is expected to require any other form of antineoplastic therapy, while on study;
has received a live vaccine within 30 days prior to the first dose of study treatment;
has received colony-stimulating factors (e.g., granulocyte colony-stimulating factor [GCSF], granulocyte-macrophage colony-stimulating factor [GM-CSF] or recombinant erythropoietin) within 28 days prior to the first dose of study treatment;
is currently receiving either strong (phenobarbital, enzalutamide, phenytoin, rifampicin, rifabutin, rifapentine, carbamazepine, nevirapine and St John's Wort) or moderate (e.g. bosentan, efavirenz, modafinil) inducers of CYP3A4 that cannot be discontinued for the duration of the study;
is currently receiving either strong (e.g., itraconazole, telithromycin, clarithromycin, protease inhibitors boosted with ritonavir or cobicistat, indinavir, saquinavir, nelfinavir, boceprevir, telaprevir) or moderate (e.g. ciprofloxacin, erythromycin, diltiazem, fluconazole, verapamil) inhibitors of cytochrome P450 (CYP)3A4 that cannot be discontinued for the duration of the study;
is unable to interrupt aspirin or other nonsteroidal anti-inflammatory drugs (NSAIDs), other than an aspirin dose≤1.3 grams per day, for at least 2 days before, during, and for at least 2 days after administration of pemetrexed;
is unable/unwilling to take folic acid, vitamin B12, and dexamethasone during administration of pemetrexed;
is currently participating in or has participated in a study of an investigational agent or has used an investigational device within 4 weeks prior to the first dose of study treatment;
has resting electrocardiogram (ECG) indicating uncontrolled, potentially reversible cardiac conditions, as judged by the investigator or has congenital long QT syndrome;
has a diagnosis of immunodeficiency or is receiving chronic systemic steroid therapy or any other form of immunosuppressive therapy within 7 days prior the first dose of study intervention;
has a known additional malignancy that is progressing or has required active treatment within the past 5 years with the exception of basal cell carcinoma of the skin, squamous cell carcinoma of the skin, superficial bladder cancer, or carcinoma in situ (e.g., breast carcinoma, cervical cancer in situ) that have undergone potentially curative therapy;
has severe hypersensitivity (≥Grade 3) to study intervention and/or any of its excipients;
has an active autoimmune disease that has required systemic treatment in past 2 years;
has a history of (noninfectious) pneumonitis/interstitial lung disease that required steroids or has current pneumonitis/interstitial lung disease;
has an active infection requiring systemic therapy;
has a known history of human immunodeficiency virus (HIV) infection;
has a known history of Hepatitis B or known active Hepatitis C virus infection;
has active tuberculosis (TB; Mycobacterium tuberculosis) and is receiving treatment;
has a history or current evidence of any condition, therapy, or laboratory abnormality that might confound the results of the study, interfere with the participant's participation for the full duration of the study, or is not in the best interest of the participant to participate, in the opinion of the treating investigator;
is considered a poor medical risk due to a serious, uncontrolled medical disorder or nonmalignant systemic disease in the opinion of the treating investigator; has a known psychiatric or substance abuse disorder that would interfere with the participant's ability to cooperate with the requirements of the study;
is unable to swallow orally administered medication or has a gastrointestinal disorder affecting absorption;
is pregnant or breastfeeding or expecting to conceive or father children within the projected duration of the study, starting with the screening visit through 180 days after the last dose of study treatment;
has had an allogenic tissue/solid organ transplant.

1. A method of treating cancer, comprising administering to a patient in need thereof a combination of:
(a) an effective amount of one or more programmed death 1 (PD-1) antagonists;
(b) an effective amount of a radiotherapy;
(c) an effective amount of one or more poly(ADP-ribose) polymerase (PARP) inhibitors; and
(d) optionally, an effective amount of one or more chemotherapies.

2. The method of claim 1, wherein each PARP inhibitor of (c) is independently selected from olaparib, niraparib, rucaparib, and talazoparib, or a pharmaceutically acceptable salt thereof.

3. The method of claim 1, wherein one PARP inhibitor of (c) is administered once or multiple times and the PARP inhibitor is olaparib, or a pharmaceutically acceptable salt thereof.

4. The method of claim 14, wherein the PD-1 antagonist is an anti-PD-1 antibody or an anti-PD-L2 antibody.

5. The method of claim 1, wherein each PD-1 antagonist of (a) is an anti-PD-1 antibody and is independently selected from pembrolizumab, nivolumab, cemiplimab, sintilimab, tislelizumab, camrelizumab and toripalimab; or each PD-1 antagonist of (a) is an anti-PD-L1 antibody and is independently selected from atezolizumab, durvalumab and avelumab.

6. The method of claim 1, wherein one PD-1 antagonist of (a) is administered once or multiple times and the PD-1 antagonist is an anti-PD-1 antibody selected from pembrolizumab and nivolumab.

7. The method of claim 6, wherein the anti-PD-1 antibody is pembrolizumab.

8. The method of claim 1, wherein the radiotherapy of (b) is a thoracic radiotherapy administered at a dose of about 10 Gy to about 100 Gy in one or more fractions.

9. The method of claim 8, wherein the radiotherapy of (b) is administered at a dose of about 20 Gy to about 80 Gy in one or more fractions.

10. The method of claim 8, wherein the radiotherapy of (b) is administered at a dose of about 60 Gy in 30 daily doses of 2 Gy.

11. The method of claim 1, wherein the one or more PD-1 antagonists of (a), the radiotherapy of (b), the one or more PARP inhibitors of (c), and the optional one or more chemotherapies of (d) are administered according to the following:
(1) a treatment phase comprising administering an effective amount of a PD-1 antagonist in combination with an effective amount of a radiotherapy and an optional effective amount of a chemotherapy; and followed by
(2) a maintenance phase comprising administering an effective amount of a PARP inhibitor.

12. The method of claim 1, wherein the one or more PD-1 antagonists of (a), the radiotherapy of (b), the one or more PARP inhibitors of (c), and the optional one or more chemotherapies of (d) are administered according to the following:
(1) a treatment phase comprising administering an effective amount of a PD-1 antagonist in combination with an effective amount of a radiotherapy and an optional effective amount of a chemotherapy; and followed by
(2) a maintenance phase comprising administering an effective amount of a PD-1 antagonist in combination with an effective amount of a PARP inhibitor.

13. The method of claim 12, comprising:
(1) a treatment phase comprising administering an effective amount of a PD-1 antagonist in combination with an effective amount of a radiotherapy and an effective amount of a chemotherapy;
wherein the radiotherapy and the chemotherapy are administered concurrently; and followed by:
(2) a maintenance phase comprising administering an effective amount of a PD-1 antagonist in combination with an effective amount of a PARP inhibitor;
wherein the PD-1 antagonist is administered once or multiple times for up to 12 months; and
wherein the PARP inhibitor is administered once or multiple times for up to 12 months.

14. The method of claim 11, wherein the PD-1 antagonist is an anti-PD-1 antibody or an anti-PD-L1 antibody.

15. The method of claim 11, wherein:
each PD-1 antagonist of the treatment phase (1) is an anti-PD-1 antibody and is selected from pembrolizumab, nivolumab and cemiplimab; and
each PD-1 antagonist of the maintenance phase (2), when present, is an anti-PD-1 antibody, and is selected from pembrolizumab, nivolumab and cemiplimab.

16. The method of claim 11, wherein:
each PD-1 antagonist of the treatment phase (1) is an anti-PD-1 antibody and is pembrolizumab;
each PD-1 antagonist of the maintenance phase (2), when present, is an anti-PD-1 antibody and is pembrolizumab;
the radiotherapy of (b) is a standard thoracic radiotherapy administered at a dose of about 20 Gy to about 80 Gy in multiple fractions; and
each PARP inhibitor of (c) is olaparib, or a pharmaceutically acceptable salt thereof.

17. The method of claim 1,
wherein chemotherapy is administered.

18. The method of claim 17, wherein the chemotherapy is selected from adriamycin, bleomycin, cisplatin, carboplatin, dactinomycin, daunorubicin, docetaxel, etoposide, irinotecan, mitomycin C, paclitaxel, pemetrexed, plicamycin, podophyllotoxin, topotecan, vincristine, and a combination of any two or more of the forgoing chemotherapies, or the chemotherapy is a platinum doublet selected from:
(1) a combination of cisplatin and pemetrexed;
(2) a combination of cisplatin and etoposide; and
(3) a combination of carboplatin and paclitaxel.

19. (canceled)

20. (canceled)

21. The method of claim 18, wherein the chemotherapy is a platinum doublet selected from:
(1) cisplatin 75 mg/m² IV and pemetrexed 500 mg/m² IV in three cycles (Day 1 in each cycle of Cycles 1-3);
(2) cisplatin 50 mg/m² IV (Days 1 and 8 in Cycles 1 and 2; Days 8 and 15 in Cycle 3) and etoposide 50 mg/m² IV in three cycles (Days 1 to 5 in Cycles 1 and 2; days 8 to 12 in Cycle 3); and
(3) carboplatin AUC 6 mg/mL/min IV with paclitaxel 200 mg/m² IV on Day 1 in Cycle 1; carboplatin AUC 2 mg/mL/min IV with paclitaxel 45 mg/m² IV on Days 1, 8, and 15 in Cycles 2 and 3.

22. The method of claim 1, comprising administering a PD-1 antagonist of (a), a radiotherapy of (b), a PARP inhibitor of (c) and a chemotherapy of (d) according to the following:
(1) a treatment phase comprising administering an effective amount of a PD-1 antagonist in combination with an effective amount of a radiotherapy and an effective amount of a chemotherapy; and followed by
(2) a maintenance phase comprising administering an effective amount of a PD-1 antagonist in combination with an effective amount of a PARP inhibitor.

23. The method of claim 22, comprising:
(1) a treatment phase comprising administering an effective amount of a PD-1 antagonist in combination with an effective amount of a radiotherapy and an effective amount of a chemotherapy;
wherein the PD-1 antagonist is administered once or multiple times;
wherein the radiotherapy is administered at a dose of about 20 Gy to about 80 Gy in one or more fractions; and
wherein the chemotherapy is administered once or multiple times; and
followed by
(2) a maintenance phase comprising administering an effective amount of a PD-1 antagonist in combination with an effective amount of a PARP inhibitor;
wherein the PD-1 antagonist is administered once or multiple times for up to 12 months; and
wherein the PARP inhibitor is administered once or multiple times for up to 12 months.

24. The method of claim 22, comprising:
(1) a treatment phase comprising administering an effective amount of a PD-1 antagonist in combination with an effective amount of a radiotherapy and an effective amount of a chemotherapy;
wherein the PD-1 antagonist is selected from pembrolizumab, nivolumab, cemiplimab, sintilimab, tislelizumab, camrelizumab and toripalimab; and
wherein the radiotherapy is a standard thoracic radiotherapy;
wherein the chemotherapy is selected from adriamycin, bleomycin, cisplatin, carboplatin, dactinomycin, daunorubicin, docetaxel, etoposide, irinotecan, mitomycin C, paclitaxel, pemetrexed, plicamycin, podophyllotoxin, topotecan, vincristine and a combination of any two or more of the foregoing chemotherapies; and followed by
(2) a maintenance phase comprising administering an effective amount of a PD-1 antagonist in combination with an effective amount of a PARP inhibitor;
wherein the PD-1 antagonist is an anti-PD-1 antibody and is selected from pembrolizumab, nivolumab and cemiplimab; and
wherein the PARP inhibitor is selected from olaparib, niraparib, rucaparib, and talazoparib, or a pharmaceutically acceptable salt thereof.

25. The method of claim 17, wherein the PD-1 antagonist of the treatment phase (1) is administered at a dose of 50 mg to 600 mg or 1-4 mg/kg once every three to six weeks.

26. The method of claim 17, wherein the PD-1 antagonist of the treatment phase (1) is pembrolizumab administered at a dose of 200 mg or 2 mg/kg IV once every three weeks, or 400 mg or 4 mg/kg IV once every six weeks.

27. (canceled)

28. The method of claim 17, wherein the radiotherapy is administered at a dose of about 20 Gy to about 80 Gy, or about 60 Gy in fractions of 30 daily doses of 2 Gy.

29. (canceled)

30. (canceled)

31. (canceled)

32. The method of claim 22, wherein the chemotherapy is selected from:
(1) cisplatin 75 mg/m² IV and pemetrexed 500 mg/m² IV in three cycles (Day 1 in each cycle of Cycles 1-3);
(2) cisplatin 50 mg/m² IV (Days 1 and 8 in Cycles 1 and 2; Days 8 and 15 in Cycle 3) and etoposide 50 mg/m² IV in three cycles (Days 1 to 5 in Cycles 1 and 2; days 8 to 12 in Cycle 3); and
(3) carboplatin AUC 6 mg/mL/min IV with paclitaxel 200 mg/m² IV on Day 1 in Cycle 1; carboplatin AUC 2 mg/mL/min IV with paclitaxel 45 mg/m² IV on Days 1, 8, and 15 in Cycles 2 and 3.

33. The method of claim 22, wherein the PD-1 antagonist of the maintenance phase (2) is administered at a dose of 100 mg to 600 mg once every three to six weeks, or 200 mg once every three weeks for up to 12 months.

34. (canceled)

35. The method of claim 22, wherein the PD-1 antagonist of the maintenance phase (2) is pembrolizumab administered at a dose of 400 mg once every six weeks for up to 12 months.

36. (canceled)

37. The method of claim 22, wherein the PARP inhibitor of the maintenance phase (2) is olaparib, or a pharmaceutically acceptable salt thereof administered at a dose of 100 mg to 600 mg twice daily.

38. (canceled)

39. (canceled)

40. The method of claim 22, comprising:
(1) a treatment phase comprising administering a PD-1 antagonist in combination with the radiotherapy and a chemotherapy;
wherein the PD-1 antagonist is administered at a dose of 100 mg to 600 mg once every three to six weeks;
wherein the radiotherapy is administered at a dose of about 20 Gy to about 80 Gy in one or more fractions;
wherein the chemotherapy is selected from cisplatin, carboplatin, etoposide, paclitaxel, pemetrexed and a combination of any two of the foregoing chemotherapies; and
administered at a dose of 10-2000 mg/m² of each chemotherapy up to 3 cycles; and followed by
(2) a maintenance phase comprising administering a PD-1 antagonist in combination with a PARP inhibitor;
wherein the PD-1 antagonist is administered at a dose of 100 mg to 600 mg once every three to six weeks in one or more cycles; and
wherein the PARP inhibitor is administered at a dose of 100 mg to 600 mg twice daily in one or more cycles.

41. The method of claim 40, comprising:
(1) a treatment phase comprising administering a PD-1 antagonist in combination with the radiotherapy and a chemotherapy;
wherein the PD-1 antagonist is pembrolizumab administered at a dose of 200 mg once every three weeks, or 400 mg once every six weeks;
wherein the radiotherapy is a standard thoracic radiotherapy administered at a dose of about 60 Gy in fractions of 30 daily doses of 2 Gy each;
wherein the chemotherapy is selected from cisplatin, carboplatin, etoposide, paclitaxel, pemetrexed and a combination of any two of the foregoing chemotherapies; and
administered at a dose of 10-2000 mg/m² of each chemotherapy up to 3 cycles; and followed by
(2) a maintenance phase comprising administering a PD-1 antagonist in combination with a PARP inhibitor;
wherein the PD-1 antagonist is pembrolizumab administered at a dose of 200 mg once every three weeks for up to 12 months, or 400 mg once every six weeks for up to 12 months; and
wherein the PARP inhibitor is olaparib, or a pharmaceutically acceptable salt thereof, administered at a dose of 300 mg twice daily for up to 12 months.

42. (canceled)

43. The method of claim 17, wherein the PD-1 antagonist, the radiotherapy and the chemotherapy of the treatment phase (1) are concurrent therapies administered on the same day or on different days, and are administered sequentially or concurrently.

44. The method of claim 17, wherein the PD-1 antagonist and the PARP inhibitor of the maintenance phase (2) are administered on the same day or on different days, and are administered sequentially or concurrently.

45. (canceled)

46. A pharmaceutical kit comprising:
(a) a PD-1 antagonist;
(b) instructions on administering a radiotherapy;
(c) a PARP inhibitor; and
(d) optionally, a chemotherapy.

47. The pharmaceutical kit of claim 46 comprising:
(a) a PD-1 antagonist selected from pembrolizumab, nivolumab, cemiplimab, sintilimab, tislelizumab, camrelizumab and toripalimab;
(b) instructions on administering a radiotherapy as part of a treatment phase;
(c) a PARP inhibitor selected from olaparib, niraparib, rucaparib, and talazoparib, or a pharmaceutically acceptable salt thereof; and
(d) a chemotherapy selected from cisplatin, carboplatin, etoposide, paclitaxel, pemetrexed and a combination of any two of the foregoing chemotherapies.

48. The pharmaceutical kit of claim 46 comprising:
(a) a PD-1 antagonist which is pembrolizumab;
(b) instructions on administering a radiotherapy as part of a treatment phase at a dose of about 20 Gy to about 80 Gy;
(c) a PARP inhibitor which is olaparib, or a pharmaceutically acceptable salt thereof; and
(d) a chemotherapy selected from:
(1) a combination of cisplatin and pemetrexed;
(2) a combination of cisplatin and etoposide; and
(3) a combination of carboplatin and paclitaxel.

49. (canceled)

50. (canceled)

51. (canceled)

52. (canceled)

53. (canceled)

54. (canceled)

55. The method of claim 1, wherein the cancer is selected from the group consisting of bladder cancer, breast cancer, colorectal cancer, hepatocellular carcinoma, melanoma, non-small cell lung cancer (NSCLC), ovarian cancer, pancreatic cancer, prostate cancer, and renal cell carcinoma.

56. (canceled)

57. (canceled)
